(19) United States
(12) Patent Application Publication
(10) Pub. No.: US 2011/0151580 A1
(43) Pub. Date: Jun. 23, 2011

(54) METHOD FOR THE DETECTION OF BREAST CANCER BY DETERMINING ALCAM AND/OR BCAM LEVELS IN A PATIENT

(75) Inventors: Eleftherios P. Diamandis, Toronto (CA); Vathany Kulasingam, Toronto (CA)

(73) Assignee: UNIVERSITY HEALTH NETWORK, Toronto, ON (CA)

(21) Appl. No.: 12/741,716

(22) PCT Filed: Jun. 6, 2008
(86) PCT No.: PCT/CA2008/001072

§ 371 Date: Mar. 9, 2011

Related U.S. Application Data
(60) Provisional application No. 60/985,861, filed on Nov. 6, 2007.

(51) Int. Cl. G01N 33/566; G01N 30/96

(52) U.S. Cl. 436/501; 422/69

(57) ABSTRACT

The present application describes biomarkers and methods useful for screening for, diagnosing or detecting the presence and severity of breast cancer in a subject. The present application also provides methods for determining the prognosis of a subject with breast cancer as well as methods for monitoring the therapeutic response to a breast cancer treatment or therapy.

### FIELD OF THE INVENTION

The present application relates to methods and compositions for screening for, detecting or diagnosing likelihood and severity of breast cancer.

### BACKGROUND OF THE INVENTION

Breast cancer is by far the most common cancer affecting women worldwide with approximately one million new cases diagnosed each year. It is a leading cause of death among women with solid tumors in North America¹. It is a disease of the middle and late ages of life, as 75% of breast cancer is diagnosed in women over the age of 50. While breast cancer is less common at a young age, younger women tend to have a more aggressive form of the disease than older women. The five-year survival rate is close to 97% when the cancer is confined to the breast². However, when breast cancer has metastasized at the time of diagnosis, the five-year survival rate is ˜23%. Gene expression patterns have been used to classify breast tumors into clinically relevant subgroups (luminal A, luminal B, basal, ERBB2-overexpressing and normal-like)^{3,4}. In general, the luminal subtypes are estrogen receptor (ER) positive and grow slowly whereas basal-type lack ER and are usually high-grade cancers that grow rapidly. Recently, the molecular taxonomy has been confirmed by protein expression profiling^{5,6}.

The main presenting features in women with symptomatic breast cancer include a lump in the breast, nipple change or discharge and skin contour changes. Historically, surveillance has included clinical history, physical examination, mammography, chest X-ray, biochemical testing and the use of tumor markers. This practice is based on the assumption that the early detection of recurrent disease leads to a better outcome. However, at present, the clinical benefit of close surveillance is unclear⁷. Currently, mammography remains the cornerstone of breast cancer screening despite its disadvantages such as high false positive and negative rates, hazardous exposure and patient discomfort^{8,9}. In addition, for women under the age of 40, mammographic screening yields a poor sensitivity of only around 33%^{10,11}. Definitive diagnosis of breast cancer requires biopsy and histopathology. In addition, the clinical course is highly variable, so it is crucial to be able to predict the course of the disease in individual patients to ensure adequate treatment and surveillance. Not all patients with breast cancer may need adjuvant treatment (e.g. approximately 70% of lymph node-negative patients are cured of their disease by surgery and radiotherapy¹²) and not all patients benefit from specific treatments. Rational disease management requires the availability of reliable prognostic and predictive markers. Currently available blood-based biomarkers are of no value in the early diagnosis of breast cancer.

Although adjuvant therapy improves patient outcome in general, at least 25-30% of women with lymph node-negative and at least 50-60% of those with lymph node-positive disease develop recurrent disease¹³. Therapy options for metastatic breast cancer include chemotherapy (e.g. anthracycline or taxane-based), hormone therapy or biological therapy (Herceptin®) combined with chemotherapy. Currently, metastatic breast cancer is regarded as incurable and thus the goal of treatment is generally palliative. In this context, the use of serial measurements of serum tumor marker(s) taken e.g. weekly, monthly, semi-annually or annually is potentially useful in deciding whether to persist in using a particular type of therapy, to terminate its use or to switch to an alternative therapy.

Carcinoembryonic antigen (CEA) and carbohydrate antigen 15-3 (CA 15-3) are the most commonly used tumor markers for breast cancer. Their levels in serum are related to tumor size and nodal involvement and are recommended for monitoring therapy of advanced breast cancer or recurrence. However, due to low diagnostic sensitivity and specificity, they are not suitable for population screening¹⁴⁻¹⁶. The CA 15-3 and BR 27.29 (also known as CA27.29) serum assays detect the same antigen, i.e. MUC1 protein and provides similar clinical information. CA 15-3 has however, been more widely investigated than BR 27.29. There are conflicting views about the value of CA 15-3 and BR 27.29 in the postoperative surveillance of patients without evidence of disease. Currently, no tumor marker exists that can be used for either screening or the early diagnosis of breast cancer. For CA 15-3, the diagnostic sensitivity of the test is 10-15%, 20-25% and 30-45% in patients with stage I, stage II and stage III disease, respectively. Furthermore, increased levels of CA 15-3 can be observed in several non-neoplastic conditions, including benign breast pathology, chronic liver disorders and immunological disorders. For CEA, the diagnostic sensitivity of the test is usually half that of CA 15-3.

Furthermore, there is no universally accepted or clinically validated definition of a clinically significant tumor marker increase. A confirmed increase of at least 25% however, is widely interpreted to signify a clinically significant increase. Based on current evidence, the National Academy of Clinical Biochemistry (NACB) Panel recommends against routine CA 15-3 (or BR 27.29) testing in asymptomatic patients following diagnosis of operable breast cancer. According to both American Society of Clinical Oncology (ASCO) and National Comprehensive Cancer Network (NCCN), CA 15-3 (or BR 27.29) should not be used alone for monitoring therapy in advanced disease. However, for patients with non-evaluable disease, both Panels state that a confirmed increase in marker concentrations suggests progressive disease. As for CA 15-3 and BR 27.29, the NACB Panel does not recommend routine use of CEA in the surveillance of patients with diagnosed breast cancer. For monitoring patients with advanced disease, CEA should not be used alone.

Tumor metastasis involves invasive growth into neighboring tissue, survival in circulation, extravasation and colonization of distant organs. Therefore, movement through tissue barriers is a pivotal step in metastasis. For this step to occur, proteolysis of extracellular matrix, remodeling of the actin cytoskeleton and selective cell adhesion interactions are all important factors. Cell adhesion molecules (CAMs) are cell surface receptors that mediate cell-cell and cell-substrate interactions¹⁷. These molecules can be grouped into four families: integrins, cadherins, selectins and the immunoglobulin superfamily (Ig-SF)¹⁸. Alterations in cellular adhesion and communication can contribute to uncontrolled cell growth. Tumor cells use adhesion molecules to cluster together and they must maintain their adhesion to each other to invade.

In this respect, ALCAM (CD166 or human melanoma metastasis clone D [MEMD]) is a type 1 transmembrane glycoprotein of the Ig-SF¹⁹. Its gene localizes to chromosome 3q13.11. The molecular weight of ALCAM is 65 kDa but with N-glycosylation at 8 putative sites, the mature ALCAM molecule has a molecular weight of 110 kDa²⁰. Five extracellular Ig domains, a transmembrane region and a short cytoplasmic tail make up the ALCAM protein that resembles E-cadherin in motif-arrangement¹⁹. ALCAM mediates both heterophilic (ALCAM-CD6 [lymphocyte cell-surface receptor]) and homophilic (ALCAM-ALCAM) cell-cell interactions²¹. The extracellular structures of ALCAM provide two structurally and functionally distinguishable modules, one involved in ligand binding (to CD6)²² and the other in avidity²³. Both modules are required for stable, homophilic ALCAM-ALCAM cell-cell adhesion²¹. Its short cytoplasmic tail does not contain any known signaling motifs. Physiologically, ALCAM is expressed in activated leukocytes and neural, epithelial and hematopoietic progenitor cells²⁴. Functionally, ALCAM has been hypothesized to act as a cell surface sensor to register local growth saturation and to regulate cellular signaling and dynamic responses¹⁷. ALCAM-CD6 interaction is required for optimal activation of T-cells.

ALCAM expression has been explored in a number of different tumor types displaying a clear up-regulation in some tumors and down-regulation in others. In addition, variable levels of ALCAM expression have been found at different stages of tumor development in the same type of malignancies. In melanoma, ALCAM has been suggested to exhibit a role in melanoma cell invasion and neoplastic progression²⁵. In prostate carcinoma, ALCAM gene was found up-regulated in high Gleason grade prostate cancers compared to benign prostatic hyperplasia cases²⁶. However, one study observed an up-regulation of ALCAM in low-grade tumors and a down-regulation in high-grade prostatic tumors²⁷. Yet, another study on prostate cancer found ALCAM to predict prostate-specific antigen (PSA) relapse²⁴. In colon cancer, using IHC, no significant correlation with patient age, tumor grade, stage or nodal status and ALCAM expression was observed, but membranous ALCAM expression correlated significantly with shortened patient survival²⁸.

There have been a few studies investigating ALCAM expression in breast cancer. Low levels of ALCAM mRNA correlated with nodal involvement, high grade and worse prognosis²⁹. In fact, low levels of ALCAM transcripts in the primary breast tumor correlated with skeletal metastases and poor prognosis³⁰. At the protein level, laser scanning cytometry and confocal microscopy showed that high levels of ALCAM correlated with small tumor diameter, low grade and the presence of hormone receptors, which supported the view that this adhesion molecule is a tumor suppressor with prognostic significance¹⁹. However, an IHC analysis showed that high cytoplasmic ALCAM expression was associated with shortened patient disease-free survival³¹. Yet a further study found that ALCAM-ALCAM interactions between breast cancer cells were important for survival in the primary tumor and that a loss of ALCAM was associated with programmed cell death³². Finally, Ihnen et al. discovered that patients with high ALCAM mRNA expression who did not receive chemotherapy tended to have a worse prognosis, suggesting that high ALCAM expression levels may be a marker for prediction of the response to adjuvant chemotherapy in breast cancer³³. Indeed, the discordant data between RNA and protein levels of ALCAM in breast cancer and even discordance among different protein expression studies suggest the need for additional research to evaluate the role of ALCAM in breast cancer.

Besides ALCAM, two additional members of this family are CD146/MUC18 and BCAM/Lutheran blood group glycoprotein (basal cell adhesion molecule)³⁴. The BCAM gene is located on chromosome 19q13.2 and is 12.5 kb long, with its cloning reported in 1994³⁵. It is the first laminin receptor that is a member of the Ig superfamily. Laminins are a family of extracellular proteins that are an integral part of all basement membranes and of the extracellular matrix proteins, only α5 chain-containing laminins are known ligands for Lu-BCAM. Lu-BCAM is a glycoprotein in which the extracellular region contains 2 variable and 3 constant Ig-like domains. Very limited information is available about the expression of BCAM in tumors and therefore the roles of BCAM in tumor progression remain unclear.

With the completion of the Human Genome Project, optimistic views were expressed that many more cancer biomarkers will be discovered through various high-throughput techniques, such as microarrays and mass spectrometry to enable early detection of breast cancer.

WO2006/016110 discloses a number of genetic markers whose expression is correlated with clinical prognosis of a given breast cancer. Six molecular signatures, made up of 12 groups of markers have been identified. The ALCAM gene has been reported to be part of a set of molecular signatures. However, this methodology consists of a plurality of genetic markers and involves the use of patient tissue in order to arrive at a conclusion regarding patient prognosis. In addition, another invention (WO2003/093443) claims to have a method for diagnosing whether an individual has breast cancer by determining whether or not there is expression of ALCAM on breast cancer cells using an anti-ALCAM antibody.

There is a need in the art for improved methods of screening, diagnosing or detecting breast cancer, particularly at an early stage.

### SUMMARY OF THE INVENTION

The present application discloses biomarkers which are differentially present in breast cancer patients compared to subjects without breast cancer. The present application provides novel methods of screening for, detecting or diagnosing breast cancer, including early stage breast cancer, using the biomarkers of the present application. In addition, the present application provides methods of predicting the prognosis of an individual having or suspected of having breast cancer as well as methods of monitoring the efficacy of a therapy used to treat breast cancer using biomarkers of the present application. Immunoassays, compositions and kits comprising the biomarkers of the present application are also provided.

An aspect of the present application is a method of screening for, diagnosing or detecting breast cancer by determining a level of an ALCAM biomarker product in a sample from a subject, wherein the sample is a biological fluid, and comparing the level in the sample with a control, wherein detecting a differential level of biomarker product between the subject and the control is indicative of breast cancer in the subject.

Another aspect of the present application is a method of screening for, diagnosing or detecting breast cancer by determining a level of a BCAM biomarker product in a sample from a subject and comparing the level in the sample to a control, wherein detecting a differential level of the biomarker product between the subject and the control is indicative of breast cancer in the subject.

A further aspect of the present application is a method of screening for, diagnosing or detecting breast cancer by determining a level of product from both an ALCAM biomarker and a BCAM biomarker in a sample from a subject and comparing each level in the sample to a control, wherein detecting a differential expression of at least one of the biomarker products between the subject and the control is indicative of breast cancer in the subject.

Yet a further aspect of the present application is a method of predicting the prognosis of a subject having or suspected of having breast cancer by determining the level of a biomarker product in a sample from the subject, where the biomarker is selected from ALCAM, BCAM and/or a combination thereof, and comparing each level of biomarker with a reference level associated with a disease outcome, the disease outcome being good prognosis, or poor prognosis, where the disease outcome associated with the reference level most similar to the level of each biomarker in the sample is the predicted prognosis. In one embodiment, an increase in ALCAM and/or BCAM is indicative of poor prognosis. In certain embodiments, the therapy comprises chemotherapy. In other embodiments, the therapy comprises a test therapy.

Yet a further aspect of the present application is a method for monitoring the therapeutic response of a subject undergoing treatment for breast cancer by determining a level of biomarker product in a first sample of the subject, the biomarker selected from the group consisting of ALCAM, BCAM and a combination thereof, determining the level of biomarker product in a subsequent sample, the subsequent sample taken subsequent to the subject receiving a treatment or therapy, and comparing the level of the biomarker product in the first sample to the level of the biomarker product in the subsequent sample, where an increase in the in the level of the biomarker product is indicative of treatment failure or a negative therapeutic response and/or a decrease in the level of the biomarker product is indicative of treatment efficacy or a positive therapeutic response. In certain embodiments, the biomarker is ALCAM. In other embodiments, the biomarker is BCAM. In yet other embodiments, the biomarkers are ALCAM and BCAM. In certain embodiment, the sample is a biological fluid. In another embodiment, the sample comprises blood, plasma, serum, a tumor, a biopsy, a nipple aspirate fluid (NAF) and/or tumor interstitial fluid (TIF). In another embodiment, the ample comprises a fresh sample, a refrigerated sample or a frozen sample. In another embodiment, the product of the biomarker is detected extracellularly. In another embodiment, the differential level of biomarker product is an increase in the sample compared to the control of at least 20% or 25%. In one embodiment, the increased level of ALCAM biomarker product indicative of breast cancer is greater than a 90% specificity cut off, or for example greater than about 62 μg/L. In another embodiment, the increased level of BCAM biomarker product indicative of breast cancer is greater than a 90% specificity cut off, or for example greater than about 32 μg/L.

In certain embodiments, the methods further comprise determining a level of at least one additional biomarker product associated with breast cancer. In yet other embodiments, the methods comprise determining the level of at least one additional biomarker product associated with breast cancer. In one embodiment, the biomarker product associated with breast cancer comprises a MUC-1 biomarker product. In one embodiment, the biomarker product associated with breast cancer comprises a CA 15-3 and/or a BR 27.29 biomarker. In certain embodiments, the level of CA15-3 is normal and/or less than about 30 U/mL. In certain embodiments, the level of CA15-3 is greater than about 30 U/mL. In another embodiment the biomarker product associated with breast cancer is a CEA biomarker product. In one embodiment, the level of CEA is less than about 5 ng/mL. In another embodiment, the level of CEA is greater than about 5 ng/mL.

In certain embodiments of the present application, the breast cancer is an early stage breast cancer. In another embodiment, the breast cancer is non-invasive, metastatic, invasive ductal carcinoma, invasive lobular carcinoma, luminal subtype, basal A-like subtype, ER+, PgR+, ER−, PgR−, PTEN−, Her2/neu amplified, and/or erbB2 amplified.

In certain embodiments, the step of determining a level of a biomarker product comprises use of isolated polypeptides that bind to ALCAM and/or BCAM biomarkers. The isolated polypeptides are antibodies. In other embodiments, the level of biomarker product is determined using an immunoassay, the immunoassay preferably being an ELISA. In yet another embodiment the biomarker products determined comprise cleaved, secreted, released or shed biomarker polypeptide products. In certain embodiments, the immunoassay is used in addition to traditional diagnostic techniques for breast cancer. Another aspect of the present application is an immunoassay for screening for, detecting or diagnosing breast cancer in a subject, determining prognosis of a subject having or suspected of having breast cancer, or monitoring therapeutic response of a subject to a breast cancer treatment, comprising an antibody that binds a biomarker of the present application immobilized to a solid support. In one embodiment the biomarker is ALCAM. In another embodiment the biomarker is BCAM. In yet another embodiment, the immunoassay comprises an antibody that binds an ALCAM biomarker and an antibody that binds a BCAM biomarker.

A further aspect of the application provides a composition comprising an agent, such as antibody, that binds an ALCAM biomarker and/or an agent that binds a BCAM biomarker. In another embodiment, the composition further comprises an agent that binds a MUC-1 and/or CEA gene product. In one embodiment, the composition comprises an agent that binds CA 15-3. In another embodiment, the composition comprises an agent that binds BR 27.29.

Another aspect of the present application is a kit for screening for detecting, or diagnosing breast cancer in a subject, determining prognosis of a subject having or suspected of having breast cancer, or monitoring the therapeutic response of a subject to a breast cancer treatment, the kit comprising in one embodiment, an antibody to an ALCAM biomarker and/or an antibody to a BCAM biomarker and instructions for use.

Other features and advantages of the present application will become apparent from the following detailed description. It should be understood, however, that the detailed description and the specific examples while indicating preferred embodiments of the present application are given by way of illustration only, since various changes and modifications within the spirit and scope of the present application will become apparent to those skilled in the art from this detailed description.

### BRIEF DESCRIPTION OF THE DRAWINGS

The present application will now be described in relation to the drawings in which:

FIG. 1 shows comparative Enzyme-Linked Immunosorbant Assays (ELISA). A ALCAM in serum of controls and breast cancer patients were measured in duplicate. B in BCAM serum of controls and breast cancer patients were measured in duplicate. The sensitivity and specificity of ALCAM and BCAM for breast cancer diagnosis is listed and the dotted lines indicate cut-offs at 90% specificity.

FIG. 2 depicts correlation data between ALCAM and BCAM with CA 15-3 levels for 35 samples. A The Spearman correlation coefficient between ALCAM (y-axis) and CA 15-3 (x-axis) was 0.63. B The Spearman correlation coefficient between BCAM (y-axis) and CA 15-3 (x-axis) was 0.56.

FIG. 3 shows ALCAM levels (y-axis) in control and subjects with low CA 15-3 (<30 units/mL) and high CA 15-3 (>30 units/mL) levels as measured by ELISA in serum. At 90% specificity for ALCAM (cutoff point of 62 μg/L) the sensitivity of the test for breast cancer diagnosis in patients where CA 15-3 is normal (<30 units/mL) is 78% (dotted line), supporting superiority of ALCAM versus CA 15-3 in terms of diagnostic sensitivity.

FIG. 4 depicts the correlation between ALCAM (x-axis) and BCAM (y-axis) for 35 samples. The Spearman correlation coefficient was 0.8162.

FIG. 5 depicts the distribution of ALCAM in the three groups (100 normal female, 50 normal male and 150 breast carcinoma samples) examined by an immunoassay specific to ALCAM. The solid horizontal line indicates the median value for each of the groups. The dotted horizontal line indicates the cut-off values to discriminate cancer from control subjects (ALCAM: 76 μg/L, 90% specificity cut-off). When comparing the ALCAM values between normal women (n=100) and patients with breast cancer (n=150) by the non-parametric Mann Whitney test (two-tailed), the medians were significantly different (median normal=60 μg/L; median cancer=74 μg/L; P<0.0001).

FIG. 6 depicts the distribution of CA 15-3 in the three groups (100 normal female, 50 normal male and 150 breast carcinoma samples) examined by an immunoassay specific to the molecule. The solid horizontal line indicates the median value for each of the groups. The dotted horizontal line indicates the cut-off values to discriminate cancer from control subjects (CA 15-3: 30 U/mL). When comparing the CA 15-3 values between normal women (n=100) and patients with breast cancer (n=150) by the non-parametric Mann Whitney test (two-tailed), the medians were significantly different (median normal=15 units/mL; median cancer=21 units/mL; P<0.0001).

FIG. 7 depicts the distribution of CEA in the three groups (100 normal female, 50 normal male and 150 breast carcinoma samples) examined by an immunoassay specific to the molecule. The solid horizontal line indicates the median value for each of the groups. The dotted horizontal line indicates the cut-off values to discriminate cancer from control subjects (CEA: 5 ng/mL). When comparing the CEA values between normal women (n=100) and patients with breast cancer (n=150) by the non-parametric Mann Whitney test (two-tailed), the medians were significantly different (median normal=1.3 μg/L; median cancer=1.9 μg/L; P=0.0003).

FIG. 8 displays receiver operating characteristic (ROC) curves for the three markers (CA 15-3, CEA, ALCAM). For a marker measured on continuous scales, a ROC curve is a plot of true positive fraction versus false positive fraction, evaluated for all possible cut-off point values.

### DETAILED DESCRIPTION OF THE INVENTION

### I. Biomarkers Associated with Breast Cancer

The present application discloses methods for detecting breast cancer using biomarkers which are differentially present, including differentially modified, expressed, secreted, released or shed in individuals having or not having breast cancer. The present inventors have used a proteomics approach to identify novel biomarkers associated with breast cancer. The inventors have demonstrated that detecting Activated Leukocyte Cell Adhesion molecule (ALCAM) and B-cell Adhesion Molecule (BCAM) biomarker products are useful for screening for, detecting or diagnosing breast cancer as well as for determining the prognosis of a subject having breast cancer. In addition, the biomarkers are useful for monitoring the therapeutic response of a patient to a breast cancer treatment or therapy. Further, the inventors have demonstrated that serum levels of ALCAM and BCAM biomarker products correlate with, and are prognostic of disease outcome in a patient with breast cancer.

The term “biomarker” as used herein can be any type of molecule that can be used to distinguish subjects with or without breast cancer. The term biomarker includes without limitation, a nucleic acid sequence including a gene, or corresponding RNA, or a polypeptide, fragment thereof, or epitope that is differentially present, including differentially modified (e.g. differentially glycosylated), expressed, secreted, released or shed in subjects with or without breast cancer. The biomarkers of the present application include for example ALCAM and/or BCAM. They can also include MUC-1, CA15-3, BR 27.29 and CEA.

The term “biomarker products” as used herein refer to gene products such as polypeptide and/or RNA products expressed by and/or corresponding to a biomarker described in the present application.

The term “RNA biomarker product” as used herein refers to RNA transcripts transcribed from biomarkers of the present application includes mRNA transcripts, and/or specific spliced variants of mRNA.

The term “polypeptide biomarker product” refers to polypeptide and/or fragments corresponding to a biomarker of the present application and includes polypeptides translated from the RNA transcripts of biomarkers described herein or known in the art associated with breast cancer. Polypeptide products include modified (e.g. post-translational modifications such as glycosylation), expressed, secreted, cleaved, released, and shed polypeptide products.

The term “ALCAM” as used herein means Activated Leukocyte Cell Adhesion molecule, also referred to as CD166, and includes, without limitation, all known ALCAM molecules including naturally occurring variants, and including those deposited in Genbank with accession numbers NM-001627 (Human ALCAM nucleic acid) and AAB59499, (human ALCAM polypeptide). ALCAM is a member of the family of cell adhesion molecules and is one of the members of a small subgroup of transmembrane glycoproteins in the immunoglobulin superfamily (IgSF)²¹.

The term an “ALCAM biomarker product” as used herein means an ALCAM gene product, including polypeptide biomarker product and fragments thereof that are differentially present, including modified, expressed, secreted, cleaved, released or shed in subjects with or without breast cancer. The ALCAM biomarker product detected is optionally full length ALCAM or a fragment thereof, including a cleaved fragment that is released from a cell, including released from a cell surface. In one embodiment, the ALCAM biomarker product is an ALCAM protein or protein fragment that is secreted, released or shed from a breast cancer cell.

The term “BCAM” as used herein means B-cell Adhesion Molecule and includes without limitation, all known BCAM molecules, including naturally occurring variants, and including those deposited in Genbank with accession numbers BC-050450 (human BCAM nucleic acid) and AAH50450 (human BCAM protein). BCAM is a laminin receptor that is a member of the immunoglobulin superfamily.

The term a “BCAM biomarker product” as used herein means a BCAM gene product, including RNA and protein product and fragments thereof that are differentially present, including modified, expressed, secreted, released or shed in subjects with or without breast cancer. The BCAM biomarker product detected is optionally full length BCAM or a fragment thereof, including cleaved fragments that are released or shed from a cell, including released or shed from a cell surface.

The term “additional biomarker product associated with breast cancer” as used herein refers to any biomarker in addition to ALCAM or BCAM that is differentially present in subjects with breast cancer and includes for example MUC1 and CEA. The additional biomarker products associated with breast cancer can have increased or decreased levels in a subject with breast cancer.

The term “MUC1” as used herein refers to a mucin-1 molecule including a MUC1 nucleic acid and/or a MUC1 polypeptide and includes without limitation, all known MUC1 molecules, including naturally occurring variants, and including those deposited in Genbank 001018016. MUC1 polypeptide is a transmembrane glycoprotein that is also known as polymorphic epithelial mucin (PEM), episialin, tumor-associated mucin, carcinoma-associated mucin, tumor-associated epithelial membrane antigen, epithelial membrane antigen (EMA), H23AG, peanut-reactive urinary mucin (PUM), breast carcinoma-associated antigen DF3, and CD227 antigen. MUC1 can be overexpressed in breast cancer in an unglycosylated form and comprises various epitopes including epitopes that are exposed in the unglycosylated form and which can be detected including for example CA 15-3 and BR 27.29. CA 15-3 and BR 27.29 are interchangeably used in the art to refer to MUC1.

The term “CA 15-3” as used herein refers to carbohydrate antigen 15-3 and/or cancer antigen 15-3 and refers to an epitope of MUC1 that is recognized by the monoclonal antibodies 115D8 and DF3. CA 15-3 is a serum marker/biomarker product that can be detected in serum.

The term “BR 27.29” which is also referred to as “CA 27.29” antigen as used herein refers to an epitope of MUC1 that is recognized by the antibodies B27.29 and DF3. BR 27.29 (and/or CA 27.29) is a serum marker/biomarker product that can be detected in serum.

The term “CEA” as used herein refers to carcinoembryoninc antigen and includes without limitation, all known CEA molecules, including naturally occurring variants, and including those deposited in Genbank (for example at NM-004363) CEA is a commonly used tumor marker for cancer. Its level in biological fluids is for example related to tumor size and nodal involvement.

### II. Methods

a) Methods of Screening for, Detecting or Diagnosing Breast Cancer

The present application discloses ALCAM and BCAM biomarkers which are differentially present, including modified, expressed, cleaved, secreted, released and/or shed in subjects with and without breast cancer. The products of the biomarkers described herein are useful for screening for, diagnosing or detecting breast cancer or an increased risk of breast cancer.

Accordingly, one aspect of the present application provides a method of screening for, diagnosing or detecting breast cancer in a subject comprising the steps:
(a) determining a level of a biomarker product in a sample from the subject wherein the biomarker is selected from the group consisting of ALCAM, BCAM and a combination thereof; and
(b) comparing the level of each biomarker product with a control, wherein detecting a differential level of one or more biomarker products between the subject and the control is indicative of breast cancer in the subject.

One embodiment, is a method of screening for, diagnosing or detecting breast cancer in a subject comprising the steps:
(a) determining a level of a biomarker product in a sample from the subject wherein the biomarker is selected from the group consisting of ALCAM, BCAM and a combination thereof; and
(b) comparing the level of each biomarker product in the sample with a control;
   wherein detecting an increased level of the biomarker product in the sample compared to the control is indicative of breast cancer in the subject. In certain embodiments, detecting an increased level of one or more biomarker products in the sample is indicative of breast cancer in the subject.

Another aspect is a method of screening for, diagnosing or detecting breast cancer in a subject comprising the steps:
(a) determining a level of an ALCAM biomarker product in a sample from the subject; and
(b) comparing the level of ALCAM biomarker product in the sample with a control;
   wherein detecting an increased level of the biomarker product in the sample compared to the control is indicative of breast cancer in the subject.

Another aspect is a method of screening for, diagnosing or detecting breast cancer in a subject comprising the steps:
(a) determining a level of BCAM biomarker product in a sample from the subject; and
(b) comparing the level of BCAM biomarker product in the sample with a control,
   wherein detecting an increased level of the biomarker product in the sample compared to the control is indicative of breast cancer in the subject.

A further aspect is a method of screening for, diagnosing or detecting breast cancer in a subject comprising the steps:
(a) determining a level of an ALCAM biomarker product and a BCAM biomarker product in a sample from the subject; and
(b) comparing the level of each biomarker product with a control, wherein detecting increased levels of one or more biomarker products between the subject and the control is indicative of breast cancer in the subject.

The phrase “screening for, diagnosing or detecting breast cancer” refers to a method or process of determining if a subject has or does not have breast cancer, or has or does not have an increased risk of developing breast cancer. Detection of increased levels of an ALCAM biomarker product and/or a BCAM biomarker product compared to control is indicative that the subject has breast cancer or an increased risk of developing breast cancer. In certain embodiments, the level of ALCAM and/or BCAM biomarker product is determined is secreted, released or shed ALCAM and/or BCAM biomarker product.

The term “an increased risk” as used herein is an increased risk relative to a control sample (e.g. a subject with control levels of ALCAM and BCAM such as control serum levels).

The term “subject” as used herein refers to any member of the animal kingdom, preferably a human being.

The term “differentially present, modified, expressed, secreted, released or shed” or “differential expression, secretion, release or shedding” as used herein refers to a difference, including an increase or a decrease, in the level of expression, secretion, release or shedding of the biomarkers described herein that can be assayed by measuring the level of expression of the products of the biomarkers, such as the difference in level of RNA expressed or polypeptides expressed of the biomarkers, and/or that can be assayed by determining the level of secreted, released or shed biomarkers, such as biomarker polypeptide product or fragments detected extracellularly, for example in serum. The term “difference in the level of expression, secretion, release or shedding” refers to an increase or decrease in the measurable expression level of a given biomarker product as measured by the amount of RNA and/or polypeptide product in a sample as compared with the measurable expression level of a given biomarker in a control or reference sample, and/or an increase or decrease in the measurable secreted, released or shed level of a given biomarker product as measured by the amount of extracellular biomarker polypeptide product, including cleaved polypeptide and/or polypeptide fragment in a sample as compared with the measurable secreted, released or shed level of a given biomarker product in a control sample. The term can also refer to an increase or decrease in the measurable level of a given biomarker in a population of samples as compared with the measurable level of a biomarker in a control population of samples. The term can also refer to an increase or decrease as compared to a control or reference level. For example the reference level is an identified level (e.g. a quantified level) above which subjects have an increased probability of having breast cancer and below which subjects have a decreased probability of having breast cancer. In one embodiment, the differential level can be compared using the ratio of the level of a given biomarker or biomarkers as compared with the level of the given biomarker or biomarkers of a control, wherein the ratio is not equal to 1.0. For example, a polypeptide is differentially present if the ratio of the level in a first sample as compared with a second sample, or control sample, or control reference level is greater than or less than 1.0. For example, a ratio of greater than 1, 1.2, 1.5, 1.7, 2, 3, 3, 5, 10, 12, 15, 20, 44 or more, or a ratio less than 1, 0.8, 0.6, 0.4, 0.2, 0.1, 0.05, 0.001 or less. In one embodiment, the increase or decrease is at least 20%, 25%, 30%, 40%, 50%, 60%, 70%, 80%, 90% or at least 100% compared with a second sample, or control sample, or control reference level. In another embodiment the differential expression, secretion, release or shedding level is measured using p-value. For instance, when using p-value, a biomarker is identified as being differentially present, including differentially present, including modified, expressed, secreted, released, or shed as between a first and second population when the p-value is less than 0.1, preferably less than 0.05, more preferably less than 0.01, even more preferably less than 0.005, the most preferably less than 0.001.

The term “level” as used herein refers to a quantity of biomarker that is detectable or measurable in a sample. The level optionally refers to a quantity that is cell associated including intracellular or extracellular where extracellular can include cell associated product levels such as cell surface expression and/or cleaved, secreted, released or shed product levels detected in a biological fluid such as serum. In a preferred embodiment, the level determined is extracellular and comprises cleaved, secreted, released, or shed biomarker polypeptide product.

The term “control” as used herein refers to a sample from an individual or a group of individuals who are either known as having breast cancer or not having breast cancer, or refers to a sample of breast cancer or non-breast cancer cells. For example, a level of biomarker product in a sample of a subject is compared to a level of biomarker product in a control, wherein the control is a sample, optionally the same sample type (e.g. both the sample and the control are serum samples), from an individual known as not having breast cancer. The control can also refer to a reference level.

The reference level is in one embodiment, a predetermined value that is related to a level of the biomarker in a group of individuals known as not having breast cancer (e.g. cutoff level). The cut-off level can be determined for a particular specificity, such as 90% specificity and/or sensitivity. For example the inventors have shown in one sample set that subjects with ALCAM biomarker product levels greater than the reference level of 62 microgram/L (90% specificity) have 91% probability of having breast cancer. In addition, for subjects with BCAM biomarker product levels greater than the 90% specificity cut off, for example above a reference level of 32 microgram/L, have 34% probability of having breast cancer.

The term “80% specificity cut-off” as used herein refers to the value or level that identifies 80% of subjects who do not have breast cancer. Similarly, the 90% and 95% cut-off is the value or level that identifies 90% or 95% of subjects who do not have breast cancer.

The term “80% sensitivity cut-off” as used herein refers to the value or level that identifies 80% of subjects who do have breast cancer. Similarly, the 90% and 95% cut-off is the value or level that identifies 90% or 95% of subjects who have breast cancer.

In one embodiment the specificity cut-off level is 90-95% or is greater than: 80%, 85%, 90%, 95%, 96%, 97%, 98% or 99%. In another embodiment, the cut-off level for ALCAM is 45-200 micrograms/L, 50-150 micrograms/L, 50-100 micrograms/L, 50-75 micrograms/L, 55-65 micrograms/L, 60-65 micrograms/L, 66-70 micrograms/L, 71-75 micrograms/L, 76-80 micrograms/L, 81-85 micrograms/L, 86-90 micrograms/L, 91-95 micrograms/L or 96-100 micrograms/L. In one embodiment the cut-off level for BCAM is 10-100 micrograms/L, 20-50 micrograms/L, 25-40 micrograms/L, or 30-35 micrograms/L. In another embodiment, the reference level is a previous level of biomarker detected in the subject.

The term “specificity” as used herein means the percentage of subjects who do not have breast cancer who are identified by the assay as negative (e.g., biomarker level is below the cutoff point) for the disorder.

The term “sensitivity” as used herein means the percentage of subjects who have breast cancer who are identified by the assay as positive (e.g. biomarker level is above the cutoff point for the disorder.

The term “breast cancer” as used herein includes any cancerous or malignant growth that begins in the breast including but not limited to non-invasive and metastatic breast cancers, ductal carcinoma in situ, lobular carcinoma in situ, invasive and/or infiltrating lobular and/or ductal carcinomas, inflammatory breast cancer, and medullary carcinoma. The term also includes breast cancers characterized as luminal subtype, basal A-like subtype, ER+, PgR+, ER−, PgR−, PTEN−, Her2/neu amplified, and/or erbB2 amplified. Breast cancer as used herein also includes different stages of breast cancer including but not limited to stage I, II (A and B), III (A, B and C) and IV.

The term “sample” as used herein refers to any biological fluid, cell or tissue sample from a subject which can be assayed for biomarker products, including ALCAM and/or BCAM gene products differentially present, including modified, expressed, secreted, released or shed, in subjects having or not having breast cancer. For example the sample optionally comprises blood, tumor biopsy, serum, plasma, nipple aspirate fluid (NAF) or tumor interstitial fluid (TIF).

In one embodiment, the sample comprises blood, plasma, serum, tumor, biopsy, nipple aspirate fluid (NAF) and/or tumor interstitial fluid (TIF). In another embodiment, the sample comprises serum, plasma and/or blood including for example fractionated blood. In a preferred embodiment, the sample comprises serum. A person skilled in the art is familiar with the techniques for obtaining a serum sample. The inventors have demonstrated that the sample can be frozen, fresh and/or refrigerated. Accordingly, in one embodiment, the sample comprises a fresh sample, a refrigerated sample or a frozen sample.

The sensitivity of the methods described herein can be improved by combining the methods described herein with at least one additional biomarker product associated with breast cancer. For example the inventors have demonstrated that the sensitivity of detecting breast cancer can be increased when determining the level of an ALCAM biomarker product is combined with determining the level of CA 15-3.

Accordingly in one embodiment, the application provides methods further comprising determining the level of at least one additional biomarker product associated with breast cancer. In one embodiment, the at least one additional biomarker product associated with breast cancer comprises a MUC-1 and/or a CEA biomarker product. In one embodiment the level of MUC-1 biomarker product is determined by determining the level of CA 15-3 and/or BR 27.29. In another embodiment, the levels of ALCAM and CA15-3 are determined and/or the levels of ALCAM and BR 27.29 are determined. In a further embodiment, the levels of BCAM or CA15-3 are determined and/or the levels of BCAM and BR 27.29 are determined. The level of the additional biomarker associated with breast cancer is compared to a control, wherein the control comprises a level in a subject without breast cancer and/or a reference level. In a further embodiment, the CA15-3 level is a normal level. In one embodiment, the CA15-3 level is less than or equal to about 30 U/ml. In another embodiment, the level of CA15-3 is greater than about 30 U/mL. In another embodiment, the biomarker products comprise ALCAM, and CEA biomarker products. In another embodiment, the biomarker products comprise BCAM and CEA biomarker products. In certain embodiments, the CEA level is less than about 5 ng/mL. In other embodiments, the level of CEA is greater than about 5 ng/mL.

The inventors have also shown that the biomarkers described herein are useful for the detection of breast cancer at early stages. The inventors have shown that determining the level of a biomarker product described herein is useful for detecting early stage breast cancer. For example the inventors demonstrate that detection of ALCAM biomarker products identifies subjects that have normal CA15-3 levels <30 U/ml. The inventors show that detection of ALCAM identifies 78% of subjects who would be missed by testing for CA15-3.

Accordingly, one aspect provides a method of screening for, diagnosing or detecting breast cancer wherein the breast cancer is early stage breast cancer. In another embodiment, an ALCAM level that is increased in comparison to control where the level of MUC1, alternatively CA 15-3 and/or BR 27.29 is normal and/or equal to or less than 30 U/mL is indicative that the patient has early stage breast cancer.

The term “early stage breast cancer” and “non-aggressive breast cancer” as used herein refers to breast cancer that is stage I or stage II. The term “advanced stage of breast cancer” and “aggressive breast cancer” as used herein refers to stage III or stage 1V breast cancer.

In one embodiment, the method of screening for, diagnosing or detecting breast cancer in a subject comprises using binding agents such as isolated polypeptides that bind polypeptide products of an ALCAM biomarker and/or BCAM biomarker or isolated nucleic acids that hybridize to RNA products of an ALCAM biomarker and/or isolated nucleic acids that hybridize to RNA products of a BCAM biomarker. Optionally, the polypeptides are antibodies and the detection assay is an immunoassay. In yet another embodiment the polypeptide products of an ALCAM biomarker and/or a BCAM biomarker determined are cleaved, secreted, released or shed biomarker polypeptide products. These are further described below.

### b) Method of Determining Prognosis

In addition to using the biomarkers of the present application for screening for, diagnosis or detection of breast cancer, biomarkers of the present application can be used for determining a prognosis of a subject having breast cancer by correlating the level of an ALCAM biomarker product and/or a BCAM biomarker product with a reference level which corresponds to a disease outcome.

Accordingly, an aspect of the present application provides a method for determining a prognosis of a subject having or suspected of having breast cancer, comprising the steps of:
(a) determining a level of a biomarker product in a sample from a subject, the biomarker selected from the group consisting of ALCAM, BCAM and a combination thereof, and
(b) comparing the level of each biomarker product with a reference level corresponding to a disease outcome, the disease outcome being good prognosis or poor prognosis
   wherein the disease outcome associated with the reference level most similar to the level of each biomarker in the sample is the predicted prognosis.

In one embodiment, an increase in ALCAM is indicative of poor prognosis. In another embodiment an increase in BCAM is indicative of poor prognosis. In yet a further embodiment, an increase in ALCAM and BCAM is indicative of poor prognosis. In other embodiments, an increase in ALCAM and/or BCAM and an increase in MUC1, determined for example by determining CA 15-3 or BR 27.29, and/or an increase in CEA is indicative of poor prognosis. In another embodiment, an ALCAM level that is increased in comparison to control where the level of MUC1, alternatively CA 15-3 and/or BR 27.29 is normal and/or where the CA 15-3 level is equal to or less than 30 U/mL is indicative that the patient has early stage breast cancer and good prognosis.

In one embodiment, the method of determining a prognosis of a subject having breast cancer comprises using binding agents such as isolated polypeptides that bind polypeptide products of an ALCAM biomarker and/or BCAM biomarker or isolated nucleic acids that hybridize to RNA products of an ALCAM biomarker and/or isolated nucleic acids that hybridize to RNA products of a BCAM biomarker. Optionally, the polypeptides are antibodies and the detection assay is an immunoassay. In yet another embodiment the polypeptide products of an ALCAM biomarker and/or a BCAM biomarker determined are cleaved, secreted, released or shed biomarker polypeptide products. These are further described below. The methods of the present application predict clinical outcomes or prognosis independently of available biomarkers such as CA 15-3.

As used herein “prognosis”, alternatively referred to as “clinical outcome” refers to an expected course of clinical disease. The prognosis provides an indication of disease progression and includes an indication of likelihood of recurrence, metastasis, death due to disease, tumor subtype or tumor type. In one embodiment the prognosis comprises a good outcome, a poor and outcome, which corresponds to a good prognosis, and a poor prognosis, respectively. A “good outcome” or a “good prognosis” as used herein refers to an increased likelihood of disease free survival for at least 60 months. A “poor outcome” or “poor prognosis” as used herein refers to an increased likelihood of relapse, recurrence, metastasis or death within 60 months.

The term “reference level” as used herein means a quantity of biomarker product which correlates with disease outcome.

It is contemplated that the methods described herein can be used in combination with other methods of determining prognosis. For example nodal status, tumor size, tumor grade, lymphatic vascular invasion, estrogen receptor, progesterone receptor and Her2-Neu status can all be used in combination with ALCAM and/or BCAM for determining prognosis.

In addition, the levels of additional biomarker products associated with breast cancer can be determined to increase the accuracy of prognosis as described elsewhere.

### c) Monitoring Therapeutic Responses to Breast Cancer Treatment

In addition to using the biomarkers described in the present application to determine prognosis of a subject having or suspected of having breast cancer, the biomarkers described herein can be used to monitor the efficacy of a breast cancer treatment or therapy.

Accordingly, another aspect provides a method of monitoring a breast cancer treatment is provided. In one embodiment, the application provides a method for monitoring the therapeutic response of subject with breast cancer comprising the steps of determining the level of an ALCAM biomarker product and/or a BCAM biomarker product in a sample such as a serum sample or a tumor extract from a subject undergoing a breast cancer treatment at an initial time point, a reference time point, as well as at a second time point after the first time point and after the initiation of the treatment, wherein detecting no change and/or a decrease in the level of the ALCAM biomarker product and/or the BCAM biomarker product in the second sample indicates treatment efficacy and/or a positive therapeutic response.

In another embodiment, the application provides a method for monitoring the therapeutic response of a subject with breast cancer comprising the steps:
(a) determining a level of biomarker product in a first sample of the subject, the biomarker selected from the group consisting of ALCAM, BCAM and a combination thereof;
(b) determining the level of biomarker product in a subsequent sample of the subject, the subsequent sample taken subsequent to the subject receiving a breast cancer treatment or therapy; and
(c) comparing the levels of the biomarker product in the first sample to the level of the biomarker product in the subsequent,
   wherein an increase in the level of the biomarker product is indicative of treatment failure and/or a negative therapeutic response.

In another embodiment, no change or a decrease in the level of the biomarker product is indicative of treatment efficacy and/or a positive therapeutic response.

The term “treatment efficacy” and/or “positive therapeutic response” means as used herein means obtaining beneficial or desired clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. For example, no change in biomarker levels can be indicative of disease stabilization and/or prevention of disease progression. “Treatment efficacy” can also mean prolonging survival as compared to expected survival if not receiving treatment.

The term “treatment failure” or “negative therapeutic response” as used here in refers to not obtaining treatment efficacy and/or a positive therapeutic response.

In one embodiment, the method of monitoring the therapeutic response of a subject undergoing treatment for breast cancer comprises polypeptides that bind to polypeptide products of an ALCAM biomarker and/or polypeptides that bind to polypeptide products of a BCAM biomarker. Optionally, the polypeptides are antibodies and the detection assay is an immunoassay. In yet another embodiment the polypeptide products of an ALCAM biomarker and/or a BCAM biomarker are cleaved, secreted released or shed. In certain embodiments, the sample comprising the biomarker polypeptide products comprises serum. In another embodiment, the method of monitoring the therapeutic response of a subject undergoing treatment for breast cancer comprises isolated using nucleic acids that hybridize to RNA products of an ALCAM biomarker and/or isolated nucleic acids that hybridize to RNA products of a BCAM biomarker. These are described further below.

The term “breast cancer treatment”, also referred to as “breast cancer therapy”, as used herein refers to any treatment that is used on a subject having or suspected of having breast cancer, including but not limited to chemotherapy.

In one embodiment, the therapy is chemotherapy. In another embodiment, the therapy is a test therapy. In yet another embodiment, the therapy is surgery.

It is contemplated that the methods described herein can be used in combination with other methods of monitoring treatment efficacy. For example CA 15-3 levels and/or the use of imaging methods such as CT scans and ultrasound may be used in combination with ALCAM and/or BCAM for monitoring treatment efficacy.

In addition, the levels of additional biomarker products associated with breast cancer can be determined to increase the accuracy of monitoring treatment response, as described elsewhere.

### III. Binding Agents for Determining Biomarker Levels

### a) Binding Agents for Detecting Biomarker Polypeptides

The level of biomarker product is optionally determined using a binding agent that specifically binds a biomarker polypeptide product. Accordingly, in one embodiment, the method of screening for, diagnosing or detecting breast cancer comprises using binding agents such as an isolated polypeptide that binds polypeptide products of the biomarkers described in the present application, wherein the isolated polypeptides are used to measure the level of expression, secretion, release or shedding of the biomarkers. In one embodiment, the method comprises using an isolated polypeptide that binds a polypeptide product of an ALCAM biomarker. In another embodiment, the method comprises using an isolated polypeptide that binds a polypeptide product of a BCAM biomarker. Yet another embodiment provides a method comprising using an isolated polypeptide that binds a polypeptide product of an ALCAM biomarker and an isolated polypeptide that binds a polypeptide product of a BCAM biomarker.

The term “isolated polypeptide” as used herein refers to a polypeptideaceous agent, such as a peptide, polypeptide or polypeptide, which is substantially free of cellular material or culture medium when produced recombinantly, or chemical precursors, or other chemicals, when chemically synthesized.

The phrase “binds a polypeptide product” as used herein refers to a binding agent such as an isolated polypeptide, that specifically binds a polypeptide product of a particular biomarker described in the present application. The polypeptide product bound is optionally a full-length biomarker polypeptide product, or a fragment that is cleaved, secreted, released or shed from a cell. The polypeptide product determined is optionally intracellular, extracellular or a combination thereof.

In one embodiment, the isolated polypeptide that binds a biomarker polypeptide product is an antibody or antibody fragment. The antibody or antibody fragment is used to determine the level of a polypeptide product of an ALCAM biomarker and/or a BCAM biomarker.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

Antibodies having specificity for a specific polypeptide, such as a polypeptide product of a biomarker described in the present application, may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody-producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor and Roder., Immunol. Today 4:72-79 (1983)), the EBV-hybridoma technique to produce human monoclonal antibodies³⁶, and screening of combinatorial antibody libraries³⁷. Hybridoma cells can be screened immunochemically for production of antibodies specifically reactive with the peptide and the monoclonal antibodies can be isolated.

In one embodiment, the binding agents, including isolated polypeptides or antibodies, are labeled with a detectable marker. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as biotin, alkaline phosphatase, beta-galactosidase or horseradish peroxidase; an imaging agent; or a metal ion.

In another embodiment, the detectable signal is detectable indirectly. For example, a secondary antibody that is specific for a biomarker described in the present application and contains a detectable label can be used to detect the biomarker.

The present application also contemplates the use of “peptide mimetics” for detecting ALCAM and/or BCAM biomarker polypeptide products. Peptide mimetics are structures which serve as substitutes for peptides in interactions between molecules (see Morgan AND Gainor. (1989), Ann. Reports Med. Chem. 24:243-252 for a review). Peptide mimetics include synthetic structures which may or may not contain amino acids and/or peptide bonds but retain the structural and functional features of binding agents specific for polypeptide products of the biomarkers described in the present application. Peptide mimetics also include peptoids, oligopeptoids³⁸

A person skilled in the art will appreciate that a number of methods can be used to determine the amount of the polypeptide product of the biomarker of the present application, including immunoassays such as Western blots, ELISA, and immunoprecipitation followed by SDS-PAGE immunocytochemistry.

Any of the methods of the present application to screen for, diagnose or detect breast cancer can be used in addition or in combination with traditional diagnostic techniques for breast cancer.

### b) Binding Agents for Detecting Biomarker Nucleic Acids

In addition to measuring the level of polypeptide products of biomarkers described in the present application, differential expression of the RNA products of the biomarkers described herein can be used to screen for, detect or diagnose breast cancer. In one embodiment, the method of screening for, diagnosing or detecting breast cancer comprises using isolated nucleic acid sequences that hybridize to a RNA product of an ALCAM biomarker. Another embodiment comprises using isolated nucleic acid sequences that hybridize to a RNA product of a BCAM biomarker. Yet another embodiment comprises using isolated nucleic acid sequences that hybridize to a RNA product of an ALCAM biomarker and isolated nucleic acid sequences that hybridize to a RNA product of a BCAM biomarker.

The term “isolated nucleic acid sequence” as used herein refers to a nucleic acid substantially free of cellular material or culture medium when produced by recombinant DNA techniques, or chemical precursors, or other chemicals when chemically synthesized. An “isolated nucleic acid” is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded. The nucleic acid sequences contemplated by the present application include isolated nucleotide sequences which hybridize to a RNA product of a biomarker, nucleotide sequences which are complementary to a RNA product of a biomarker of the present application, nucleotide sequences which act as probes, or nucleotide sequences which are sets of ALCAM specific primers and/or BCAM specific primers.

The term “hybridize” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology³⁹. For example, 6.0× sodium chloride/sodium citrate (SSC) at about 45° C., followed by a wash of 2.0×SSC at 50° C. may be employed.

The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log10 [Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology³⁹ and in Molecular Cloning, a Laboratory Manual⁴⁰.

The term “primer” as used herein refers to a nucleic acid sequence, whether occurring naturally as in a purified restriction digest or produced synthetically, which is capable of acting as a point of synthesis of when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art. The term “biomarker specific primers” as used herein refers a set of primers which can produce a double stranded nucleic acid product complementary to a portion of one or more RNA products of the biomarkers described in the present application or sequences complementary thereof.

The term “probe” as used herein refers to a nucleic acid sequence that will hybridize to a nucleic acid target sequence. In one example, the probe hybridizes to a RNA product of the biomarker of the present application or a nucleic acid sequence complementary to the RNA product of the biomarker of the present application. The length of probe depends on the hybridize conditions and the sequences of the probe and nucleic acid target sequence. In one embodiment, the probe is at least 8, 10, 15, 20, 25, 50, 75, 100, 150, 200, 250, 400, 500 or more nucleotides in length.

A person skilled in the art will appreciate that a number of methods can be used to measure or detect the level of RNA products of the biomarkers of the present application within a sample, including microarrays, RT-PCR (including quantitative RT-PCR), nuclease protection assays and northern blots.

It is contemplated that the methods described herein can be used in combination with other methods of screening for, diagnosing or detecting breast cancer. For example, the methods are optionally used in combination with other biomarkers such as CA 15-3 and CEA.

### IV. Immunoassays

An immunoassay is optionally used to detect biomarker polypeptide products. The inventors further developed a sandwich immunoassay for detecting ALCAM and BCAM biomarker products. The inventors used a mouse anti-human ALCAM antibody as the coating antibody and a biotinylated goat anti-human ALCAM antibody as the detection antibody to develop a sandwich immunoassay for detection of ALCAM biomarker. Similarly, the inventors used a mouse anti-human BCAM antibody as the coating antibody and a biotinylated goat anti-human BCAM antibody as the detection antibody, to develop a sandwich immunoassay for detection of BCAM biomarker.

Accordingly, one aspect provides an immunoassay for screening for, detecting or diagnosing breast cancer in a subject, determining prognosis of a subject suspected of having breast cancer, and/or monitoring the therapeutic response of a subject to a breast cancer treatment, the immunoassay comprising an antibody immobilized to a solid support and a detection antibody. In one embodiment, the immobilized antibody is an anti-human ALCAM antibody and the detection antibody is a biotinylated anti-human ALCAM antibody. In another embodiment, the immobilized antibody is an anti-human BCAM antibody and the detection antibody is a biotinylated anti-human BCAM antibody. In yet another embodiment, the immunoassay comprises anti-human ALCAM and an anti-human BCAM antibodies.

### V. Compositions

Another aspect of the application relates to compositions for determining the levels of biomarker products described herein. In one embodiment, the composition comprises an agent that binds an ALCAM biomarker and/or an agent that binds a BCAM biomarker. In another embodiment the composition comprises at least two detection agents wherein each agent binds one or more biomarker products, wherein the biomarker products comprise ALCAM, BCAM, MUC1 and/or CEA. The composition comprises in one embodiment, a suitable carrier, diluent, or additive as are known in the art.

The term “agent” as used herein refers to any molecule or compound that can bind to a biomarker product described herein, including polypeptides such as antibodies, nucleic acids and peptide mimetics.

In one embodiment the agent comprises a polypeptide. In another embodiment, the polypeptide is an antibody and/or an antibody fragment for example, an antibody described herein. In another embodiment, the agent is a nucleic acid that binds or hybridizes a biomarker product, for example a nucleic acid described herein. In a further embodiment, the agent is a peptide mimetic that binds a biomarker product described herein.

In another embodiment, the composition further comprises an agent that binds a MUC-1 and/or CEA biomarker product. In another embodiment, the agent that binds the MUC-1 biomarker product comprises an agent that binds CA 15-3 and/or an agent that binds BR 27.29.

### VI. Kits

Another aspect of the present application is a kit for screening for detecting, or diagnosing breast cancer in a subject, determining prognosis of a subject having breast cancer, and/or monitoring the therapeutic response of a subject to a breast cancer treatment. In one embodiment, the kit comprises an agent, for example an antibody to an ALCAM biomarker and/or an antibody to a BCAM biomarker and instructions for use.

In one embodiment, the application provides a kit for detecting a biomarker comprising:

a) an agent that binds a biomarker product selected from the group consisting of ALCAM or BCAM and a combination thereof; and
b) instructions for use.
In one embodiment, the kit comprises an agent that binds the biomarker product ALCAM. In another embodiment, the kit comprises an agent that binds the biomarker product BCAM, In another embodiment, the kit further comprises an agent that binds a MUC-1 and/or CEA biomarker product. In another embodiment, the agent that binds MUC-1 binds CA 15-3 or BR 27.29. In a further embodiment the kit comprises an agent that binds ALCAM and an agent that binds CA15-3. In another embodiment, the kit comprises an agent is an antibody or a fragment thereof that specifically binds the polypeptide biomarker product.

In another embodiment, the kit comprises an isolated nucleic acid of an ALCAM biomarker and/or an isolated nucleic acid of a BCAM biomarker and instructions for use. In yet another embodiment, the kit comprises an agent that binds or hybridizes a nucleic acid biomarker product. In one embodiment, the agent is a probe that specifically hybridizes the biomarker nucleic acid product.

The following non-limiting examples are illustrative of the present application:

### EXAMPLES

### Example 1

### Materials & Methods

### Cell Lines

The breast epithelial cell line MCF-10A, and the breast cancer cell lines BT-474 and MDA-MB-468 were purchased from the American Type Culture Collection (ATCC), Rockville, Md. MCF-10A was maintained in Dulbecco's modified Eagle's medium and F12 medium (DMEM/F12) supplemented with 8% fetal bovine serum (FBS), epidermal growth factor (20 ng/mL), hydrocortisone (0.5 μg/mL), cholera toxin (100 ng/mL) and insulin (10 μg/mL). BT-474 and MDA-MB-468 were maintained in phenol-red-free RPMI 1640 culture medium (Gibco) supplemented with 8% FBS. All cells were cultured in a humidified incubator at 37° C. and 5% CO₂ in tissue culture T-75 cm² flasks.

### Cell Culture

Approximately 30×10⁶ cells were seeded individually into six 175 cm² tissue culture flasks per cell line. After 2 days, the RPMI or DMEM/F12 media were discarded and the cells rinsed twice with 1× phosphate buffered saline (PBS). Following this, 30 mL of Chemically Defined Chinese Hamster Ovary (CDCHO) serum-free medium (Gibco), supplemented with glutamine (8 mM) (Gibco) was added and the flasks were incubated for an additional 24 hours. The conditioned media (CM) were collected and spun down to remove cellular debris. CM were then frozen at −80° C. until further use. A 1 mL aliquot was taken at the time of harvest to measure for total protein (Bradford assay), lactate dehydrogenase (LDH) and human kallikreins 5, 6 and 10 (KLK5, KLK6, KLK10) via ELISA. The adhered cells were trypsinized and counted using a hemocytometer. This procedure was repeated several times for reproducibility. In addition, 30 mL of the culture media (RPMI 1640 and DMEM/F12) were subjected to the same conditions as above, with no cells added, and used for comparison. For the MDA-MB-468 cell lysate experiment, at the end of 24 hours in SFM, the adhered cells were lyzed using a French Press (Thermo Electron), where the cells are sheared by forcing them through a narrow space. Total protein was measured and 400 μg of protein from the lysate was added to 60 mL of CDCHO medium and processed in the same manner as the CM. The cell lysate experiment was performed in duplicate.

### Sample Preparation

Two 30 mL CM were combined (total of 60 mL) for each cell line, creating 3 biological replicates per cell line, and dialyzed using a molecular weight cut-off membrane of 3.5 kDa. The CM was dialyzed in 5 L of 1 mM ammonium bicarbonate solution overnight, at 4° C. with two buffer changes. The dialyzed CM was poured equally into two 50 mL conical tubes. The CM was frozen and lyophilized to dryness. The lyophilized sample was denatured using 8 M urea and reduced with dithiothreitol (DTT, final concentration 13 mM; Sigma). Following reduction, the sample was alkylated with 500 mM iodoacetamide (Sigma) and desalted using a NAPS column (GE Healthcare). The sample was lyophilized and trypsin (Promega) digested (1:50, trypsin:protein concentration) overnight in a 37° C. water bath. Following this, the peptides were lyophilized to dryness.

### Strong Cation Exchange Liquid Chromatography

The trypsin-digested dry sample was resuspended in 120 μL of mobile phase A (0.26 M formic acid in 10% acetonitrile). The sample was directly loaded onto a PolySULFOETHYL A™ column (The Nest Group, Inc.) containing a hydrophilic, anionic polymer (poly-2-sulfoethyl aspartamide). A 200 Å pore size column with a diameter of 5 μm was used. A one hour fractionation procedure was performed using a high performance liquid chromatography (HPLC) system (Agilent 1100). A linear gradient of 0.26 M formic acid in 10% acetonitrile as the running buffer and 1 M ammonium formate added as the elution buffer was used. The eluent was monitored at a wavelength of 280 nm. Forty fractions, 200 μL each, were collected every minute after the start of the elution gradient. These 40 fractions were pooled into 8 combined fractions (each pool consisting of 5 fractions) and lyophilized to ˜200 μL.

### Mass Spectrometry (LC-MS/MS)

The 8 pooled fractions per replicate per cell line were C₁₈ extracted using a ZipTip_{C18} pipette tip (Millipore; catalogue # ZTC18S096) and eluted in 4 μL of 68% ACN, made up of Buffer A and Buffer B (90% ACN, 0.1% formic acid, 10% water, 0.02% TFA). 80 μL of Buffer A (95% water, 0.1% formic acid, 5% ACN, 0.02% TFA) was added and 40 μL were injected onto a 2 cm C18 trap column (inner diameter 200 μm). The peptides were eluted from the trap column onto a resolving 5 cm analytical C18 column (inner diameter 75 μm) with an 8 micron tip (New Objective). The LC set-up was coupled online to a 2-D Linear Ion Trap (LTQ, Thermo Inc) mass spectrometer using a nanoelectrospray ionization source (ESI) in data-dependent mode. Each pooled fraction was run on a 120 minute gradient. The eluted peptides were subjected to tandem mass spectrometry (MS/MS). DTAs were created using the Mascot Daemon@ (v2.16) and extract_msn. The parameters for DTA creation were: min. mass 300, max. mass 4000, automatic precursor charge selection, min. peaks 10 per MS/MS scan for acquisition and a min. scans per group of 1.

### Mass Spectrometry Data Analysis

The resulting raw mass spectra from each pooled fraction were analyzed using Mascot® (Matrix Science, London, UK; version 2.1.03) and X!Tandem® (GPM Manager, version 2.0.0.4) search engines on the non-redundant IPI Human database V3.16 (62000+ entries). Up to one missed cleave was allowed and searches were performed with fixed carbamidomethylation of cysteines and variable oxidation of methionine residues. A fragment tolerance of 0.4 Da and a parent tolerance of 3.0 Da were used for both search engines, with trypsin as the digestion enzyme. This operation resulted in 8 DAT files (Mascot) and 8 XML files (X!Tandem) for each replicate sample per cell line. Scaffold® (version Scaffold-01_{—}05_{—}19, Proteome Software Inc., Portland, Oreg.) was used to validate MS/MS based peptide and protein identifications. Peptide identifications were accepted if they could be established at greater than 95.0% probability as specified by the PeptideProphet® algorithm⁴¹. Protein identifications were accepted if they could be established at greater than 80.0% probability and contained at least identified peptide. Protein probabilities were assigned by the ProteinProphet® algorithm⁴². Proteins that contained similar peptides and could not be differentiated based on MS/MS analysis alone were grouped to satisfy the principles of parsimony. The DAT and XML files for each cell line plus their respective negative control files (RPMI or DMEM culture media only) were inputted into Scaffold to cross-validate Mascot and X!Tandem data files. Each replicate sample was designated as one biological sample containing both DAT and XML files in Scaffold and searched with MudPit option clicked. The results obtained from Scaffold were processed using an in-house developed program that generated the protein overlaps between samples. Each protein identified was assigned a cellular localization based on information available from Swiss-Prot, Genome Ontology (GO), Human Protein Reference Database (HPRD) and other publicly available databases. To calculate the false-positive error rate, the individual fractions were analyzed using the “sequence-reversed” decoy IPI Human V3.16 database by Mascot and X!Tandem and data analysis was performed as mentioned above.

### Spectral Counting

Using the number of total spectra output from Scaffold, the inventors identified the differentially expressed proteins using spectral counting. Common peptides among proteins were grouped and proteins containing more than 10% of their total spectra from negative control samples were removed and one excel file containing total proteins identified and their presence (defined by spectral counts) in the 3 cell lines were generated. A normalization criterion was applied to normalize the spectral counts so that the values of the total spectral counts per sample were similar. An average of the spectral counts was generated for each cell line (based on the triplicate samples). The sum of the 3 variances for the cell lines, an indicator of the variance within each cell line, was calculated. The variance of the average spectral counts for each cell line revealed the variability between the cell lines. ANOVA (Fisher test) was performed to obtain the ratio of the “between sample variance” to the “within sample variance”. Apparent fold-changes were calculated when possible.

### Total Protein Assay and LDH Measurements

Total protein was quantitated in the CM using a Coomassie (Bradford) protein assay reagent (Pierce). All samples were loaded in triplicates on a microtiter plate and protein concentrations were estimated by reference to absorbances obtained for a series of bovine serum albumin (BSA) standard protein dilutions. Lactate dehydrogenase (LDH), an intracellular enzyme which if found in the CM is an indicator of cell death, was measured using an enzymatic assay based on lactate to pyruvate conversion and parallel production of NADH from NAD. The production of NADH was measured by spectrophotometry at 340 nm using an automated method (Roche Modular system).

### Quantification of ALCAM and BCAM—Immunoassay

96-well polystyrene plates were first coated with 250 ng/well of ALCAM or BCAM monoclonal antibody (R&D). After overnight incubation, the plates were washed and loaded with 50 μL of serum or standards and 50 μL of an assay buffer for 1 hour. After washing the plate, 100 μL of a biotinylated ALCAM or BCAM monoclonal antibody (R&D) was added, creating a sandwich-type assay, and the plates were incubated for an additional 1 hour with gentle shaking. After washing, alkaline phosphatase-conjugated streptavidin was added and incubated for 15 min and washed. Finally diflunisal phosphate (DFP) and terbium-based detection was performed, essentially as described by Christopoulos et al.⁴³. Fluorescence was measured with a time-resolved fluorometer, the Cyberfluor 615 Immunoanalyzer (MDS Nordion, Kanata, ON, Canada). The calibration and data reduction were performed automatically. A total of 35 breast serum samples with known amounts of CA15-3 were evaluated.

### Results and Discussion

The pathogenic signaling pathways involved during the process of cancer initiation and progression are not confined to the cancer cell itself but are rather extended to the tumor-host interface. This interface can be thought of as a dynamic environment in which fluctuating information flows between the tumor cells and the normal host tissue. Recognizing that cancer is a product of the proteomic tissue microenvironment has several significant implications. For example, the tumor-host interface can generate enzymatic cleavage and shedding, and sharing of growth factors. Therefore, it is conceivable that either the tumor itself or its microenvironment could be sources for biomarkers that would ultimately be shed into the serum proteome, allowing for early disease detection and for monitoring therapeutic efficacy.

The inventors have performed a proteomics study to identify breast cancer biomarkers using a cell culture approach and later validated the identified candidate breast cancer biomarkers in relevant biological fluids. Sampling the secretome representing breast cancer progression using a cell culture system (MCF-10A, BT474 and MDA-B-468) and qualitative proteomic analysis involving mass spectrometry resulted in a number of candidate molecules that were evaluated for their potential to be circulating breast cancer biomarkers in serum using an Enzyme-Linked Immunosorbant Assay (ELISA) or other quantitative proteomic methodologies.

MCF-10A, a basal B subtype, with intact p53, was derived by spontaneous immortalization of breast epithelial cells from a patient with fibrocystic disease and it has been used extensively as a normal control in breast cancer studies⁴⁴. These cells do not survive when implanted subcutaneously into immunodeficient mice. BT474, a luminal subtype, obtained from a stage II localized solid tumor, is positive for ER and progesterone receptor (PgR), which represent 50-60% of all breast cancer cases⁴⁵. This cell line also displays amplification of Her-2/neu or erbB-2—which represents 30% of all breast cancer cases⁴⁶. Her2/neu is a cell membrane surface-bound tyrosine kinase involved in signal transduction, leading to cell growth and differentiation. Its over-expression is associated with a high risk of relapse and death⁴⁶ and is the target of the therapeutic monoclonal antibody Herceptin⁴⁷. Finally, MDA-MB-468, a basal A-like subtype, obtained from a pleural effusion of a stage 1V patient⁴⁸, is ER and PgR negative (15-25% of breast cancer) and PTEN negative (30% of breast cancer)^{49,50}.

These cell lines were cultured in serum-free media (SFM) to ensure that the collected conditioned media (CM) contain no other extraneous proteins, except for the secreted or shed proteins from the cancer cells. By collecting and concentrating large volumes of CM produced from cell lines representing semi-normal (MCF-10A), non-invasive (BT474) and metastatic origins (MDA-MB-468), the secreted and shed proteins would accumulate in the CM, thereby facilitating their identification through mass spectrometry (MS). Comparative proteomic analysis of the CM obtained from MCF-10A, BT474 and MDA-MB-468 identified over 600, 500 and 700 proteins, respectively. A large portion of the proteins was present in CM from all 3 cell lines; however, a significant portion contained proteins that were unique to each of the cell lines. Among these were the internal control proteins, human kallikreins 5, 6 and 10 being identified by MS and ELISA in MDA-MB-468 cells, at a concentration ranging from 2-50 μg/L. Members of the human kallikrein family (KLKs) have been implicated in the process of carcinogenesis and the application of kallikreins as biomarkers for diagnosis and prognosis are currently being investigated. Kallikreins are secreted enzymes that encode for trypsin-like or chymotrypsin-like serine proteases⁵¹. Prostate-specific antigen (PSA; KLK3), belonging to the family of human tissue kallikreins, and human kallikrein 2 (KLK2) currently have important clinical applications as prostate cancer biomarkers⁵². In addition to the control proteins, various proteases, receptors, protease inhibitors, cytokines and growth factors were identified.

Cellular localization, biological function and Unigene analyses were performed for the shortened list of candidate breast cancer biomarkers consisting of extracellular, membrane and unclassified proteins. A significant degree of overlap was observed among the proteins identified in this study using a cell culture model and other studies using relevant biological fluids such as nipple aspirate fluid (NAF) and tumor interstitial fluid (TIF). Finally, spectral counting analysis revealed promising molecules to investigate further for both understanding the disease and as potential biomarkers for breast cancer.

The following criteria were applied to select the most promising molecules to investigate further. The top 100 secreted and membrane-bound proteins from spectral counting analysis were selected. PubMed searches were performed on the 100 proteins to identify molecules that have not been studied with respect to breast cancer in the serum. The proteins that met this criterion were again filtered by searching patent websites to select only those candidate breast cancer biomarkers that were novel and not previously examined with respect to breast cancer in the serum. Further selection involved selecting candidates that were expressed exclusively or preferentially in the early or advanced stages of cancer, and whether the molecules have been known to participate in pathways related to cancer initiation and progression. This resulted in two promising candidate molecules which were investigated further: 1) activated leukocyte cell adhesion molecule (ALCAM) and 2) B-cell adhesion molecule (BCAM).

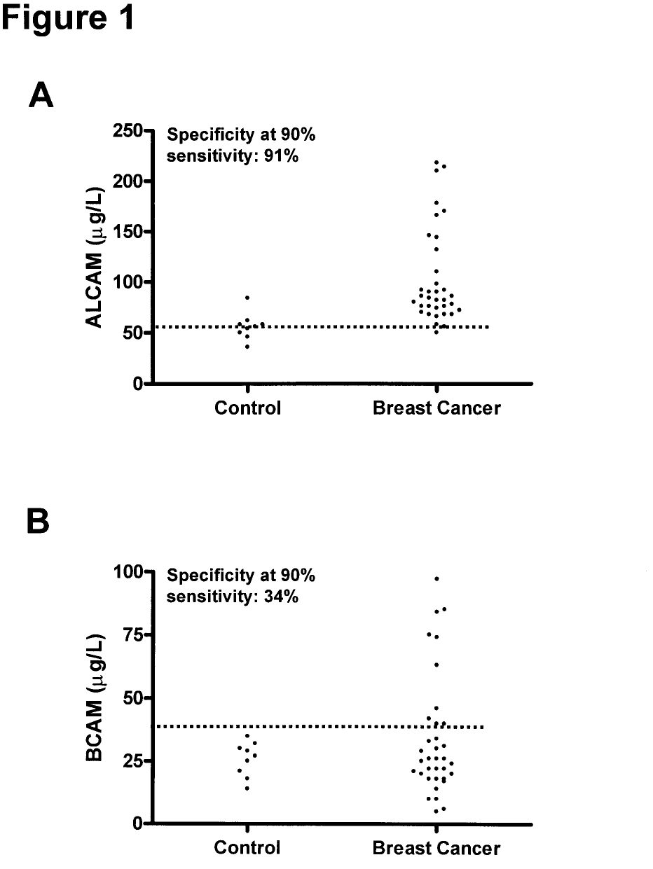

ALCAM (SwissProt ID: □13740) is a membrane bound protein that has been found in the plasma (Human Plasma Proteome database) and has not been reported in NAF or in TIF. It is a 583 amino acid long protein with previous associations to other cancer types. The inventors' spectral counting data revealed that ALCAM was not present in MCF-10A (semi-normal cell line) but was expressed in BT474 (localized) with a normalized spectral count of 42 and in MDA-MB-468 (metastatic) with a normalized spectral count of 12; yielding an F-ratio of 3. Furthermore, a relative fold change of 44 was observed for this protein when comparing BT474/MCF-10A and a fold change of 12 was observed when comparing MDA-MB-468/MCF-10A. Using a mouse anti-human ALCAM antibody (R&D) as the coating antibody and a biotinylated goat anti-human ALCAM antibody (R&D) as the detection antibody, a sandwich immunoassay was developed for this candidate in-house. Using the ELISA assay for ALCAM, the levels of ALCAM in serum of controls and breast cancer patients were measured in duplicate (FIG. 1A). The CA 15-3 levels of the breast cancer patients were measured.

Similarly, BCAM (SwissProt ID: P50895) is a plasma membrane protein that has not been reported in the plasma or in NAF or TIF previously. It is a 628 amino acid long protein that may mediate intracellular signaling. The obtained spectral counting data revealed that BCAM was not present in MCF-10A but was expressed in BT474 with a normalized spectral count of 37 and in MDA-MB-468 with a normalized spectral count of 9; yielding an F-ratio of 6. Using a mouse anti-human BCAM antibody (R&D) as the coating antibody and a biotinylated goat anti-human BCAM antibody (R&D) as the detection antibody, a sandwich immunoassay was developed for this candidate breast cancer biomarker. Using the ELISA assay for BCAM, the levels of BCAM in the same sample set as ALCAM were measured in duplicate (FIG. 1B).

Correlation Between ALCAM and BCAM with CA 15-3 Levels

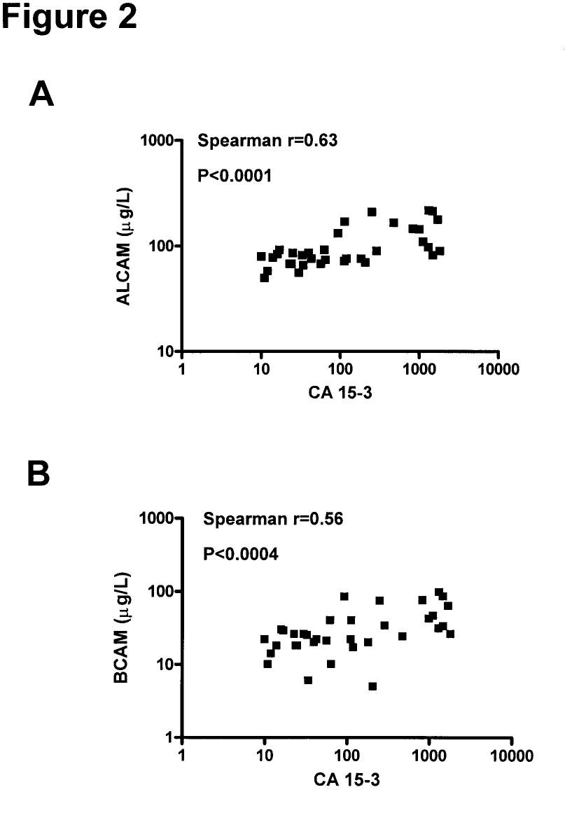

The Spearman correlation coefficient between ALCAM (y-axis) and CA 15-3 (x-axis) was 0.63 (95% confidence interval of 0.37-0.80) for 35 samples with a P-value of <0.0001 (FIG. 2A).

The Spearman correlation coefficient between BCAM (y-axis) and CA 15-3 (x-axis) was 0.56 (95% confidence interval of 0.27-0.76) for 35 samples with a P-value of <0.0004 (FIG. 2B).

### Sensitivity and Specificity for ALCAM and BCAM

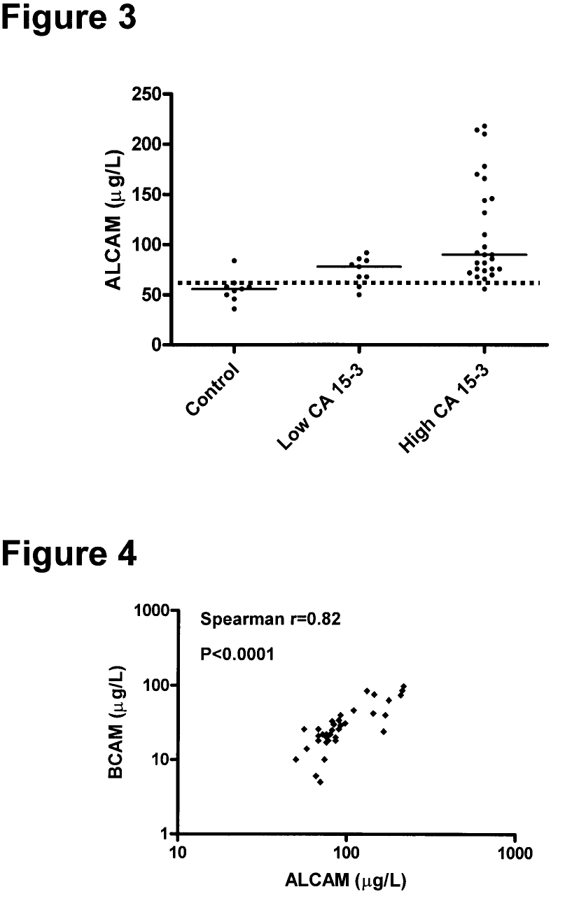

For ALCAM, at 90% specificity (cutoff point of 62 μg/L) the sensitivity for breast cancer diagnosis (all stages) was 91%. For BCAM, at 90% specificity (cutoff point of 32 μg/L) the sensitivity for breast cancer diagnosis (all stages) was 34%. Especially for ALCAM, the sensitivity of the test for breast cancer diagnosis in patients where CA 15-3 is normal (<30 U/mL) is 78%. This means that ALCAM can identify a considerable number of patients (78%) who will all be missed by CA 15-3 testing (FIG. 3). At cut-off point 62 μg/L, 8/9 control subjects fall below this point (−90% specificity). At this cut-off point (dotted line), 32/35 cancer patients fall above this point (−91% sensitivity). At this cut-off point, 7/9 low CA 15-3 cancer patients fall above this point (78%). FIG. 3 shows ALCAM levels (y-axis) in control and subjects with low CA 15-3 and high CA 15-3 levels. The levels of ALCAM were measured using an ELISA while the levels of CA 15-3 were measured using a commercial assay provided by Roche Diagnostics.

Correlation of ALCAM with BCAM Levels

The Spearman correlation coefficient between BCAM (y-axis) and ALCAM (x-axis) was 0.8162 (95% confidence interval of 0.66-0.90) for 35 samples with a P-value of <0.0001.

The results provide strong evidence that ALCAM and/or BCAM are useful for breast cancer diagnosis, prognosis or for monitoring therapeutic efficacy (FIG. 4).

### Example 2

### Materials and Methods

Applicable methods and materials as described in Example 1 were used.

### Patients and Specimens

The clinical material used consisted of 150 serum samples from primary breast cancer patients (ages 34 to 82 years; median, 62 years), 100 serum samples from normal, apparently healthy women (ages 24 to 56 years; median, 40 years), and as an additional control, 50 serum samples from normal healthy men (ages 23 to 61 years; median, 48 years). The samples from primary breast cancer patients were from untreated individuals collected prior to surgery. Histologically, 94 were classified as invasive ductal carcinoma and/or multifocal invasive ductal carcinoma, 24 as invasive lobular carcinoma and/or multifocal invasive lobular carcinoma and 32 as either invasive ductal carcinoma+invasive lobular carcinoma, invasive ductal carcinoma with various aspects, lobular carcinoma in situ, medullary carcinoma or other. Histologic classification was based on the World Health Organization of breast tumors recommendation. Patients with disease of clinical stages 1 to 3 were represented in this study. Of the 150 primary breast carcinoma patients, 32 were stage 1, 57 were stage 2A or 2B, 27 were stage 3A or 3B and stage information was not available for the remaining 34. Clinical grades 1, 2 and 3, corresponding to 26, 62 and 56 patients, respectively, were included in this study. The characteristics of the breast cancer patients in terms of tumor diameter, lymph node status, menopausal status and hormone receptor status are described later. Serum samples, obtained from Venice, Italy, from all patients were stored at −80° C. until further analysis.

### Measurement of ALCAM, CA 15-3 and CEA in Serum

The concentration of ALCAM in serum was measured by using a highly sensitive and specific non-competitive “sandwich-type” ELISA, developed by the inventors. The assay is based on mouse monoclonal antibody capture and biotinylated mouse monoclonal detection antibody (both obtained from R&D Systems, Minneapolis, Minn.). The assay has a detection limit of 0.05 μg/L and a dynamic range of up to 10 μg/L. Precision was less than 10% within the measurement range. Assay parameters such as stability, linearity, cross-reactivity, recovery and reproducibility were examined. Serum samples were analyzed in triplicate with inclusion of two quality control samples in every run. In addition, CA 15-3 and CEA were measured using a commercially available automated ELISA kit (Elecsys CA 15-3 and CEA Immunoassay, respectively; Roche Diagnostics, Indianapolis, Ind.). The upper limit of normal for CA 15-3 for this method is 30 U/mL and for CEA is 5 ng/mL.

### Data Analysis and Statistics

The relationships between biomarkers and patient and tumor characteristics were examined with the Kruskal-Wallis test, a nonparametric method for examining differences among multiple groups. Spearman's rank correlation coefficient was used to assess the correlations among biomarkers. Logistic regression was performed to calculate the odds ratio (OR) that defines the relation between biomarkers and case or control status. OR were calculated on log-transformed biomarkers and were represented with their 95% confidence interval (95% CI) and two-sided p-values.

To further evaluate the diagnostic or prognostic usefulness of the markers for dichotomous classification, receiver operating characteristic (ROC) curve analysis was considered. If by convention larger values of a biomarker are associated with adverse outcome, a cut-off point is used to define a positive marker-based test result, i.e., positive if the marker value exceeds some cut-off point. For a marker measured on continuous scales, a ROC curve is a plot of true positive fraction versus false positive fraction, evaluated for all possible cut-off point values. For binary outcome, i.e., response to chemotherapy, the ROC curve quantifies the discriminatory ability of a marker for separating cases from controls. The standard deviations of the area under the curve (AUC) and the differences between AUCs are computed with the U-statistic of DeLong et al⁵³, or the bootstrap re-sampling method.

For each ROC curve, the AUC was calculated, which ranges from 0.5 (for a non-informative marker) to 1 (for a perfect marker) and corresponds to the probability that a randomly selected case has a higher marker value than a randomly selected control. Bootstrap method was used to calculate the confidence intervals for AUC.

The ROC analysis was first conducted on individual markers and then in combination, to explore the potential that a marker panel can lead to improved performance. An algorithm that renders a single composite score using the linear predictor fitted from a binary regression model was considered. This algorithm has been justified to be optimal under the linearity assumption⁵⁴ in the sense that ROC curve is maximized (i.e., best sensitivity) at every threshold value. Since an independent validation series was not available for this study, the predictive accuracy of the composite scores was evaluated based on re-sampling of the original data. All analyses were performed using Splus 8.0 software (Insightful Corp., Seattle Wash.).

### ALCAM ELISA Assay Development

A robust sandwich-type ELISA using two monoclonal antibodies specific for the human ALCAM molecule was developed. To ensure that the immunoassay was suitable for measuring clinical serum samples, the recovery, reproducibility, linearity, cross-reactivity and serum sample stability were examined. Recombinant human ALCAM protein was added into the general diluent (control), normal serum (male and female) and into serum of breast cancer patients at different concentrations, and measured with the ALCAM immunoassay. A recovery of 90-100% was observed in these samples. The assay also showed negligible cross-reactivity to another adhesion molecule of the Ig-SF, B-cell adhesion molecule²¹, displayed excellent linearity with serial dilutions and showed <10% CV for intra- and inter-assay variability studies. Finally, the design of the stability study consisted of collecting serum at different time points (2 weeks, 4 weeks and fresh samples) and storing them at 4° C., −20° C. and −80° C. ALCAM levels were measured in these samples using the immunoassay. No difference was observed among the samples stored at the different temperature conditions and among the different time point collections, compared to the freshly obtained samples.

Association of Biomarkers with Age

Since cases and controls were not matched for age, marker values differed by age. The comparisons between cases and controls were based on data from females only. While no change with age was observed for CA 15-3 concentrations, the level of CEA appeared to increase with age for both cases and controls. With respect to ALCAM, there was a trend for marker level to increase with age for cases but not for controls. However, there was no correlation between ALCAM levels and age of normal women. This suggests that the difference in age between cases and controls is not a confounding factor in this study.

### Correlations Among Biomarkers

Spearman's rank correlation coefficients were used to assess the correlations among markers for female controls and cases, respectively, and the results are listed in Table 1. CEA appeared to be weakly correlated with ALCAM in both cases (Spearman r=0.371, p<0.001) and controls (Spearman r=0.348, p=0.001), whereas CA15-3 was weakly correlated with ALCAM among cases only (Spearman r=0.2, p=0.015).

Association of Biomarkers with Tumor Characteristics for Cases

The association of ALCAM, CA 15-3 and CEA with patient and tumor characteristics such as age, tumor diameter, ER and PgR status, grade, histology, ratio of lymph node positive (Ipos) and total lymph nodes (Itot), menopausal status, and stage were examined. A significant association was obtained for the following clinicopathologic variables: age (<=50, 51-60, 61-70 and >70), menopausal status (pre- and post-menopausal), and stage (I, II, III). The distributions of each marker in cases for these variables are given in Table 2. Post-menopausal women displayed higher values of CEA and ALCAM (all p<0.001). As well, levels of ALCAM were not significantly associated with stage whereas CEA and CA15-3 were.

Association of Biomarkers with Breast Cancer

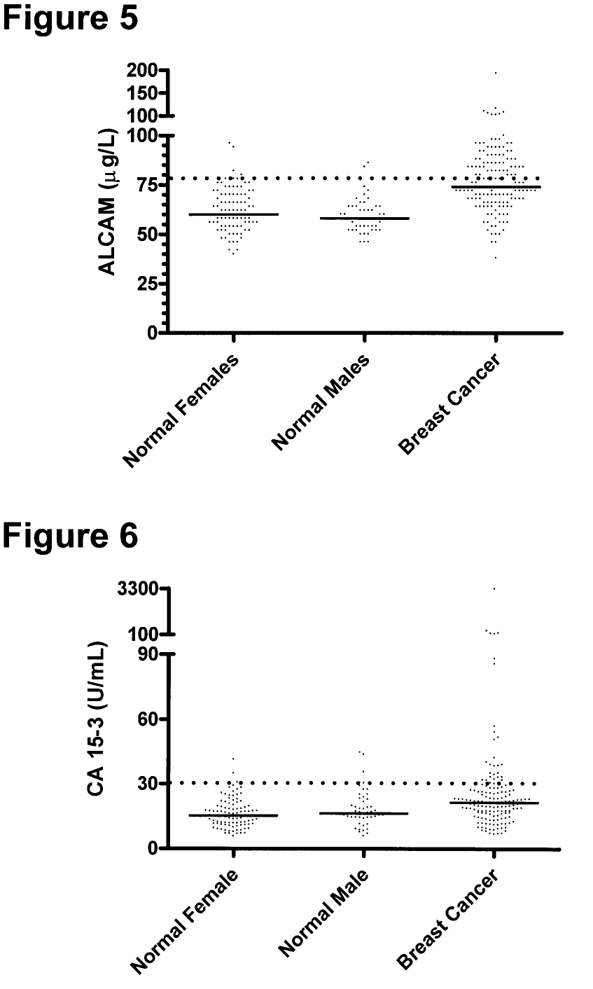

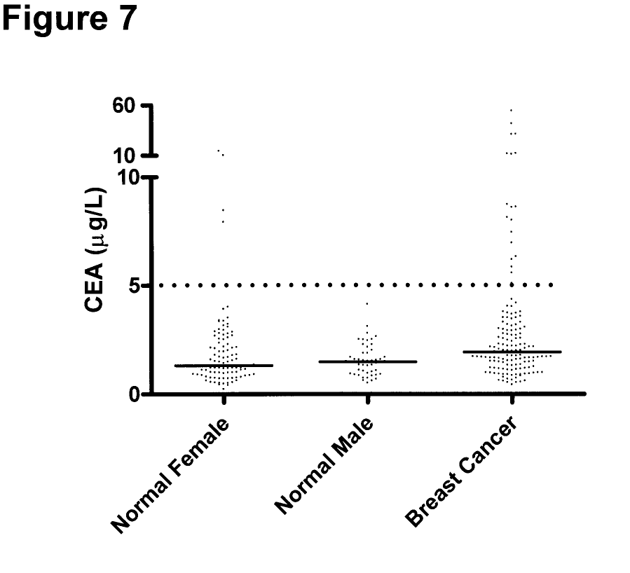

The distributions of the 3 markers, as measured by immunoassays, in cases and controls, are shown in FIGS. 5, 6 and 7 (ALCAM, CA 15-3 and CEA, respectively). Distributions of the patients with breast cancer differed from controls (female or male) for ALCAM, but to a lesser degree for the other two markers. The median values of males and females were similar for all 3 markers. When comparing the ALCAM values between normal women (n=100) and patients with breast cancer (n=150) by the non-parametric Mann Whitney test (two-tailed), the medians were significantly different (median normal=60 μg/L; median cancer=74 μg/L; P<0.0001). For CA 15-3, the medians were significantly different (median normal=15 units/mL; median cancer=21 units/mL; P<0.0001). Lastly for CEA, the medians were different (median normal=1.3 μg/L; median cancer=1.9 μg/L; P=0.0003). The association of the markers with cancer was further considered with linear regression models of logarithm-transformed marker values as a function of clinical status (cancer vs non-cancer; females only) and age. Adjusting for age, the mean levels of log(CA15-3) and log(ALCAM) were significantly higher in cancer; levels of log(CEA) did not differ between cancer and controls.

Logistic regression models were also considered to further characterize the associations between markers and breast cancer, adjusting for age. Similar to the results from linear regression, the two individual markers, CA 15-3 (OR=1.12, 95% CI [1.04, 1.19]) and ALCAM (OR=1.42, 95% CI [1.14, 1.77]) were found to univariately predict breast cancer, but this was not the case for CEA (OR=0.99, 95% CI [0.95, 1.05]). In a logistic regression model, which included age and all three markers, CA15-3 and ALCAM were found to independently predict breast cancer. Results from the logistic regression models are given in Table 3.

### The Diagnostic Values of the Three Markers

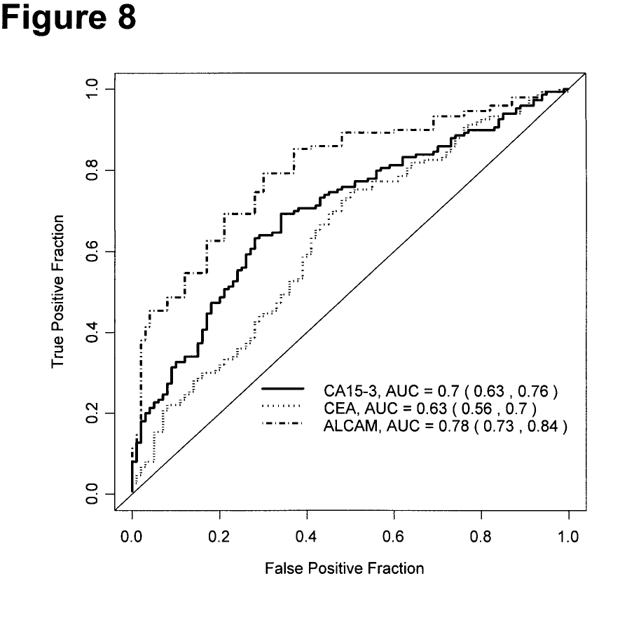

ROC curve analysis (FIG. 8) was used to quantify the diagnostic value of the three markers. All three markers have AUC significantly better than 0.5, with ALCAM having the best performance (AUC=0.78, 95% CI [0.73, 0.84]). The superiority of ALCAM over the other two markers was also evident when sensitivities at fixed values of 90% and 80% specificities, respectively were considered (Table 4). For example, at specificity of 80%, ALCAM yielded a sensitivity of 60%, compared with 48% for CA15-3. Likewise, at 90% specificity, ALCAM displayed higher sensitivity than CA 15-3 and CEA. Combining CA15-3 and ALCAM, based on the linear predictors from a logistic regression model, yielded a ROC curve with an AUC of 0.81 (bootstrap 95% CI [0.75, 0.87]). Combining CA15-3, ALCAM and CEA did not result in any improvement in ROC curves compared to CA15-3 and ALCAM. Re-sampling methods which aimed to adjust for over-fitting⁵⁵ did not yield substantially different results.

The finding of decreased levels of ALCAM in breast cancer tissue compared to normal breast tissue is not contradictory to results of elevated levels of ALCAM in serum of breast cancer patients. It is possible that ALCAM levels decrease in tissue but one elevated in serum. For example, although PSA gene transcription is down-regulated in prostate cancer, PSA protein levels in the circulation of prostate cancer patients increase due to disruption of the anatomic barriers between the glandular lumen and capillaries. Concomitant to early-stage prostate cancer is the loss of basal cells, disruption of cell attachment, degradation of the basement membrane, initiation of lymphangiogenesis⁵⁶ and loss of the polarized structure and luminal secretion by tumor cells. Consequently, PSA levels in the serum can rise to 4-10 Late-stage prostate cancer is characterized by invasion of tumor cells into the stromal layers and the circulation, and total loss of glandular organization. This situation allows for considerable amounts of PSA to leak into the bloodstream, where levels typically range from 10 to 1000 μg/l.

The inventors are the first to report presence of ALCAM in serum of breast cancer patients. Until now, all studies regarding ALCAM expression have been performed at the transcript level or using IHC or confocal microscopy. The present inventors developed a robust and highly sensitivity immunoassay to measure ALCAM in biological fluids.

A number of potential reasons exist for observing elevated levels of adhesion molecules such as ALCAM in cancer patients versus normal individuals. First, increased homotypic intercellular adhesion (due to elevated levels of these molecules) may favor the metastatic process since cell aggregates, rather than single cells breaking away from the primary tumor, have a greater chance of survival in the circulation and of lodging in other organs⁵⁷. Second, it is known that cell adhesion is necessary for the metastatic spread of cancer cells to new organs (secondary tumor establishment)⁵⁸. As well, overproduction of adhesion molecules may disrupt the normally operative intercellular adhesion forces, allowing more cell movement and the adoption of a less ordered tissue architecture⁵⁹. As an illustration, a substance that has been studied extensively as a marker for breast cancer is CEA. CEA is a member of the immunoglobulin supergene family and is expressed in a large variety of secretory tissues^{60,61}. Interestingly, expression of CEA is increased in colon carcinomas and it may be important to processes of intercellular recognition^{62,63}. It has been suggested that this might either result in disturbance of normal intercellular adhesion or provide advantages in further steps of metastasis⁵⁹ such as conceivably facilitating establishment of a secondary tumor^{58,64}. Without wishing to be bound by theory, these factors may be true for ALCAM.

Given that ALCAM is a transmembrane protein with an extracellular domain, it is very likely that membrane shedding may lead to elevated levels of ALCAM in circulation. MMP-2, a metalloproteinase involved in degrading cell-cell connections, has been shown to be elevated in serum of breast cancer patients and its levels correlate with poor prognosis^{65,56}. Again, without wishing to be bound by theory, it is possible that a putative substrate for MMP-2 is ALCAM. Therefore, it is probable that an increase in MMP-2 or other proteases (such as the kallikreins) may result in increased shedding of ALCAM into the circulation.

The present data provides evidence that serum ALCAM represents a novel biomarker for breast cancer. This biomarker displays higher diagnostic sensitivity for breast cancer than the currently used tumor markers CA 15-3 and CEA (Table 4). Moreover, as a result of the moderate correlation between ALCAM and CA 15-3 (Table 1), there are patients with normal levels of CA 15-3 (<30 U/mL) who have elevated ALCAM levels. In fact, among the 120/150 cancer patients examined who displayed normal levels of CA 15-3, 48 of them (40%) had elevated levels of ALCAM (values of 78 μg/L or greater; the cut-off for 95% specificity). For this reason, CA 15-3 measurements will benefit from combining ALCAM measurements, to increase the diagnostic sensitivity of each of the markers alone. As well, assuming a 95% specificity, the statistical power of the inventor's work (n=>100 for both control and cases) will allow the detection of a 20% difference between mean values of ALCAM levels in breast cancer patients and controls. The difference between the ALCAM means in this study was >20%, within the power of the methods described herein.

Accordingly, serum ALCAM concentration represents a novel biomarker for breast carcinoma. Further, the combination of ALCAM with CA 15-3 improved the diagnostic sensitivity. The availability of a reliable immunoassay, such as the one developed herein, for measuring serum ALCAM may in addition to establishing the clinical usefulness of this marker, also clarify the biological roles of ALCAM in breast cancer.

TABLE 1
Spearman's Rank Correlation Coefficients among 3 Markers for
Female Controls and Cases

| | Female Controls | | | Cases | | |
|---|---|---|---|---|---|---|
| | CA15-3 | CEA | ALCAM | CA15-3 | CEA | ALCAM |
| CA15-3 | 1 | | | 1 | | |
| CEA | −0.091 | 1 | | 0.161* | 1 | |
| ALCAM | 0.082 | 0.348* | 1 | 0.2* | 0.371* | 1 |

*p < 0.05

Table 1 highlights the Spearman's rank correlation coefficients among the three markers for female controls and cases examined. CEA is weakly correlated with ALCAM in both cases (Spearman r=0.371, p<0.001) and controls (Spearman r=0.348, p=0.001), whereas CA15-3 is weakly correlated with ALCAM among cases only (Spearman r=0.2, p=0.015).

TABLE 2
Marker distributions by tumor characteristics for cases

| | # of | CA15-3 | | CEA | | ALCAM | |
|---|---|---|---|---|---|---|---|
| | Patients | Median | Q31* | Median | Q31* | Median | Q31* |
| Age | | | | | | | |
| <=50 | 36 | 20.36 | 5.11 | 1.59 | 0.59 | 66.00 | 7.00 |
| 51-60 | 34 | 21.78 | 6.06 | 1.82 | 0.99 | 77.00 | 11.50 |
| 61-70 | 40 | 18.90 | 4.34 | 2.02 | 0.75 | 75.00 | 9.00 |
| 70+ | 40 | 22.92 | 6.12 | 2.62 | 0.96 | 82.00 | 8.25 |
| p value** | | 0.31 | | 0.01 | | <0.001 | |
| Meno- | | | | | | | |
| pausal | | | | | | | |
| status | | | | | | | |
| pre | 30 | 21.30 | 5.35 | 1.03 | 0.56 | 66.00 | 6.00 |
| post | 103 | 20.61 | 6.06 | 2.14 | 0.98 | 78.00 | 9.50 |
| p value** | | 0.92 | | <0.001 | | <0.001 | |
| Stage | | | | | | | |
| I | 32 | 17.20 | 5.93 | 1.48 | 0.61 | 72.00 | 11.25 |
| II | 57 | 19.46 | 4.66 | 1.75 | 0.86 | 74.00 | 8.00 |
| III | 27 | 23.40 | 10.32 | 2.47 | 1.10 | 72.00 | 11.50 |
| p value** | | 0.003 | | 0.004 | | 0.88 | |

*Q31, semi-interquartile range: computed as one half the difference between the 75^{th} percentile (Q3) and the 25^{th} percentile (Q1)
**p value: computed from global nonparametric Kruskal-Wallis test for testing the association between a marker and a clinical variable

Table 2 depicts the marker distributions by tumor characteristics for cases. The association of ALCAM, CA 15-3 and CEA with patient and tumor characteristics such as age, tumor diameter, ER and PgR status, grade, histology, ratio of lymph node positive (Ipos) and total lymph nodes (Itot), menopausal status, and stage were examined. Post-menopausal women displayed higher values of CEA and ALCAM (all p<0.001). As well, levels of ALCAM were not significantly associated with stage whereas CEA and CA15-3 were.

TABLE 3
Results from logistic regression models

| | |
|---|---|
| Univariate* | Multivariate** |

| Marker | OR | 95% CI | OR | 95% CI |
|---|---|---|---|---|
| CA15-3 | 1.12 | (1.04, 1.19) | 1.09 | (1.02, 1.18) |
| CEA | 0.99 | (0.95, 1.05) | 0.94 | (0.89, 1.00) |
| ALCAM | 1.42 | (1.14, 1.77) | 1.39 | (1.09, 1.78) |

*logistic model with logarithm of the marker and age as predictors.
**logistic model with logarithm of all three markers and age as predictors.
OR, odds ratio;
CI, confidence interval.

Table 3 displays the results from logistic regression models used to further characterize the associations between markers and breast cancer, adjusting for age. Two individual markers, CA 15-3 (OR=1.12, 95% CI [1.04, 1.19]) and ALCAM (OR=1.42, 95% CI [1.14, 1.77]) univariately predicted breast cancer, but this was not the case for CEA (OR=0.99, 95% CI [0.95, 1.05]). In a logistic regression model, which included age and all three markers, CA15-3 and ALCAM independently predicted breast cancer.

TABLE 4
ROC analysis for biomarkers

| | | | Sensitivity | | | |
|---|---|---|---|---|---|---|
| | | | 90% | | 80% | |
| Marker | AUC | 95% CI | Specificity | 95% CI | Specificity | 95% CI |
| CA15-3 | 0.70 | (0.64, 0.76) | 0.32 | (0.19, 0.44) | 0.48 | (0.32, 0.63) |
| CEA | 0.63 | (0.56, 0.70) | 0.22 | (0.12, 0.31) | 0.32 | (0.22, 0.41) |
| ALCAM | 0.78 | (0.73, 0.84) | 0.47 | (0.38, 0.57) | 0.60 | (0.48, 0.73) |
| Combined* | 0.81 | (0.75, 0.87) | 0.52 | (0.39, 0.64) | 0.67 | (0.54, 0.80) |

Combined*: linear combination of CA15-3 and ALCAM

Table 4 summarizes the ROC analysis for biomarkers. All three markers have AUC significantly better than 0.5, with ALCAM having the best performance (AUC=0.78, 95% CI [0.73, 0.84]). The superiority of ALCAM over the other two markers was considering sensitivities at fixed values of 90% and 80% specificities, respectively. For example, at specificity of 80%, ALCAM yielded a sensitivity of 60%, compared with 48% for CA15-3. Likewise, at 90% specificity, ALCAM displayed higher sensitivity than CA 15-3 and CEA. Combining CA15-3 and ALCAM, based on the linear predictors from a logistic regression model, yielded a ROC curve with an AUC of 0.81 (bootstrap 95% CI [0.75, 0.87]). Combining CA15-3, ALCAM and CEA did not result in any improvement in ROC curves compared to CA15-3 and ALCAM. Re-sampling methods which aimed to adjust for over-fitting did not yield substantially different results.

### FULL CITATIONS FOR REFERENCES REFERRED TO IN THE SPECIFICATION

1. van Diest, P. J., van der, W. E., & Baak, J. P. Prognostic value of proliferation in invasive breast cancer: a review. J. Clin. Pathol. 57, 675-681 (2004).
2. Jemal, A. et al. Cancer statistics, 2004. CA Cancer J. Clin. 54, 8-29 (2004).
3. Sorlie, T. et al. Repeated observation of breast tumor subtypes in independent gene expression data sets. Proc. Natl. Acad Sci. U.S.A 100, 8418-8423 (2003).
4. Sorlie, T. et al. Gene expression patterns of breast carcinomas distinguish tumor subclasses with clinical implications. Proc. Natl. Acad. Sci. U.S.A 98, 10869-10874 (2001).
5. Abd El-Rehim, D. M. et al. High-throughput protein expression analysis using tissue microarray technology of a large well-characterised series identifies biologically distinct classes of breast cancer confirming recent cDNA expression analyses. Int. J. Cancer 116, 340-350 (2005).
6. Jacquemier, J. et al. Protein expression profiling identifies subclasses of breast cancer and predicts prognosis. Cancer Res. 65, 767-779 (2005).
7. Emens, L. A. & Davidson, N. E. The follow-up of breast cancer. Semin. Oncol. 30, 338-348 (2003).
8. Leitch, A. M. Controversies in breast cancer screening. Cancer 76, 2064-2069 (1995).
9. Esserman, L. et al. Improving the accuracy of mammography: volume and outcome relationships. J. Natl. Cancer Inst. 94, 369-375 (2002).
10. Mincey, B. A. & Perez, E. A. Advances in screening, diagnosis, and treatment of breast cancer. Mayo Clin. Proc. 79, 810-816 (2004).
11. Antman, K. & Shea, S. Screening mammography under age 50. JAMA 281, 1470-1472 (1999).
12. Morris, A. D. et al. Breast-conserving therapy vs mastectomy in early-stage breast cancer: a meta-analysis of 10-year survival. Cancer J. Sci. Am. 3, 6-12 (1997).
13. Bernard-Marty, C., Cardoso, F., & Piccart, M. J. Facts and controversies in systemic treatment of metastatic breast cancer. Oncologist. 9, 617-632 (2004).
14. Lumachi, F. & Basso, S. M. Serum tumor markers in patients with breast cancer. Expert. Rev. Anticancer Ther. 4, 921-931 (2004).
15. Khatcheressian, J. L. et al. American society of clinical oncology 2006 update of the breast cancer follow-up and management guidelines in the adjuvant setting. J. Clin. Oncol. 24, 5091-5097 (2006).
16. Diamandis, E. P., Fritsche, H. A., Lilja, H., Chan, D. W., & Schwartz, M. K. Tumor Markers Physiology, Pathobiology, Technology, and Clinical Applications 33-63 AACC Press, Washington, D.C. (2002).
17. Swart, G. W., Lunter, P. C., Kilsdonk, J. W., & Kempen, L. C. Activated leukocyte cell adhesion molecule (ALCAM/CD166): signaling at the divide of melanoma cell clustering and cell migration? Cancer Metastasis Rev. 24, 223-236 (2005).
18. Johanning, G. L. Modulation of breast cancer cell adhesion by unsaturated fatty acids. Nutrition 12, 810-816 (1996).
19. Jezierska, A. et al. Activated Leukocyte Cell Adhesion Molecule (ALCAM) is associated with suppression of breast cancer cells invasion. Med. Sci. Monit. 12, BR245-BR256 (2006).
20. Denzinger, T. et al. Isolation, primary structure characterization and identification of the glycosylation pattern of recombinant goldfish neurolin, a neuronal cell adhesion protein. J. Mass Spectrom. 34, 435-446 (1999).
21. Swart, G. W. Activated leukocyte cell adhesion molecule (CD166/ALCAM): developmental and mechanistic aspects of cell clustering and cell migration. Eur. J. Cell Biol. 81, 313-321 (2002).
22. Bowen, M. A. & Aruffo, A. Adhesion molecules, their receptors, and their regulation: analysis of CD6-activated leukocyte cell adhesion molecule (ALCAM/CD166) interactions. Transplant. Proc. 31, 795-796 (1999).
23. van Kempen, L. C. et al. Molecular basis for the homophilic activated leukocyte cell adhesion molecule (ALCAM)-ALCAM interaction. J. Biol. Chem. 276, 25783-25790 (2001).
24. Kristiansen, G. et al. Expression profiling of microdissected matched prostate cancer samples reveals CD166/MEMD and CD24 as new prognostic markers for patient survival. J. Pathol. 205, 359-376 (2005).
25. van Kempen, L. C. et al. Activated leukocyte cell adhesion molecule/CD 166, a marker of tumor progression in primary malignant melanoma of the skin. Am. J. Pathol. 156, 769-774 (2000).
26. Stamey, T. A. et al. Molecular genetic profiling of Gleason grade 4/5 prostate cancers compared to benign prostatic hyperplasia. J. Urol. 166, 2171-2177 (2001).
27. Kristiansen, G. et al. ALCAM/CD166 is up-regulated in low-grade prostate cancer and progressively lost in high-grade lesions. Prostate 54, 34-43 (2003).
28. Weichert, W., Knosel, T., Bellach, J., Dietel, M., & Kristiansen, G. ALCAM/CD166 is overexpressed in colorectal carcinoma and correlates with shortened patient survival. J. Clin. Pathol. 57, 1160-1164 (2004).
29. King, J. A. et al. Activated leukocyte cell adhesion molecule in breast cancer: prognostic indicator. Breast Cancer Res. 6, R478-R487 (2004).
30. Davies, S. R. et al. Expression of the cell to cell adhesion molecule, ALCAM, in breast cancer patients and the potential link with skeletal metastasis. Oncol. Rep. 19, 555-561 (2008).
31. Burkhardt, M. et al. Cytoplasmic overexpression of ALCAM is prognostic of disease progression in breast cancer. J. Clin. Pathol. 59, 403-409 (2006).
32. Jezierska, A., Matysiak, W., & Motyl, T. ALCAM/CD166 protects breast cancer cells against apoptosis and autophagy. Med. Sci. Monit. 12, BR263-BR273 (2006).
33. Ihnen, M. et al. Predictive impact of activated leukocyte cell adhesion molecule (ALCAM/CD 166) in breast cancer. Breast Cancer Res. Treat. (2008).
34. Kikkawa, Y. & Miner, J. H. Review: Lutheran/B-CAM: a laminin receptor on red blood cells and in various tissues. Connect. Tissue Res. 46, 193-199 (2005).
35. Campbell, I. G. et al. Molecular cloning of the B-CAM cell surface glycoprotein of epithelial cancers: a novel member of the immunoglobulin superfamily. Cancer Res. 54, 5761-5765 (1994).
36. Roder, J. C., Cole, S. P., & Kozbor, D. The EBV-hybridoma technique. Methods Enzymol. 121, 140-167 (1986).
37. Huse, W. D. et al. Generation of a large combinatorial library of the immunoglobulin repertoire in phage lambda. Science 246, 1275-1281 (1989).
38. Simon, R. J. et al. Peptoids: a modular approach to drug discovery. Proc. Natl. Acad. Sci. U.S.A 89, 9367-9371 (1992).
39. Molecular Biology 6.3.1-6.3.6 John Wiley & Sons, New York (1989).
40. Sambrook. Molecular Cloning, a Laboratory Manual, 3, Cold Spring Harbor Laboratory Press, (1989).
41. Keller, A., Nesvizhskii, A. I., Kolker, E., & Aebersold, R. Empirical statistical model to estimate the accuracy of peptide identifications made by MS/MS and database search. Anal. Chem. 74, 5383-5392 (2002).
42. Nesvizhskii, A. I., Keller, A., Kolker, E., & Aebersold, R. A statistical model for identifying proteins by tandem mass spectrometry. Anal. Chem. 75, 4646-4658 (2003).
43. Christopoulos, T. K. & Diamandis, E. P. Enzymatically amplified time-resolved fluorescence immunoassay with terbium chelates. Anal. Chem. 64, 342-346 (1992).
44. Soule, H. D. et al. Isolation and characterization of a spontaneously immortalized human breast epithelial cell line, MCF-10. Cancer Res. 50, 6075-6086 (1990).
45. Lasfargues, E. Y., Coutinho, W. G., & Redfield, E. S. Isolation of two human tumor epithelial cell lines from solid breast carcinomas. J. Natl. Cancer Inst. 61, 967-978 (1978).
46. Slamon, D. J. et al. Human breast cancer: correlation of relapse and survival with amplification of the HER-2/neu oncogene. Science 235, 177-182 (1987).
47. Shak, S. Overview of the trastuzumab (Herceptin) anti-HER2 monoclonal antibody clinical program in HER2-overexpressing metastatic breast cancer. Herceptin Multinational Investigator Study Group. Semin. Oncol. 26, 71-77 (1999).
48. Cailleau, R., Olive, M., & Cruciger, Q. V. Long-term human breast carcinoma cell lines of metastatic origin: preliminary characterization. In Vitro 14, 911-915 (1978).
49. She, Q. B., Solit, D., Basso, A., & Moasser, M. M. Resistance to gefitinib in PTEN-null HER-overexpressing tumor cells can be overcome through restoration of PTEN function or pharmacologic modulation of constitutive phosphatidylinositol 3′-kinase/Akt pathway signaling. Clin. Cancer Res. 9, 4340-4346 (2003).
50. Panigrahi, A. R. et al. The role of PTEN and its signalling pathways, including AKT, in breast cancer; an assessment of relationships with other prognostic factors and with outcome. J. Pathol. 204, 93-100 (2004).
51. Borgono, C. A. & Diamandis, E. P. The emerging roles of human tissue kallikreins in cancer. Nat. Rev. Cancer 4, 876-890 (2004).
52. Rittenhouse, H. G., Finlay, J. A., Mikolajczyk, S. D., & Partin, A. W. Human Kallikrein 2 (hK2) and prostate-specific antigen (PSA): two closely related, but distinct, kallikreins in the prostate. Crit. Rev. Clin. Lab Sci. 35, 275-368 (1998).
53. DeLong, E. R., DeLong, D. M., & Clarke-Pearson, D. L. Comparing the areas under two or more correlated receiver operating characteristic curves: a nonparametric approach. Biometrics 44, 837-845 (1988).
54. McIntosh, M. W. & Pepe, M. S. Combining several screening tests: optimality of the risk score. Biometrics 58, 657-664 (2002).
55. Zheng, Y. et al. A multiparametric panel for ovarian cancer diagnosis, prognosis, and response to chemotherapy. Clin. Cancer Res. 13, 6984-6992 (2007).
56. Stacker, S. A., Achen, M. G., Jussila, L., Baldwin, M. E., & Alitalo, K. Lymphangiogenesis and cancer metastasis. Nat. Rev. Cancer 2, 573-583 (2002).
57. Updyke, T. V. & Nicolson, G. L. Malignant melanoma cell lines selected in vitro for increased homotypic adhesion properties have increased experimental metastatic potential. Clin. Exp. Metastasis 4, 273-284 (1986).
58. Sommers, C. L. The role of cadherin-mediated adhesion in breast cancer. J. Mammary. Gland. Biol. Neoplasia. 1, 219-229 (1996).
59. Benchimol, S. et al. Carcinoembryonic antigen, a human tumor marker, functions as an intercellular adhesion molecule. Cell 57, 327-334 (1989).
60. Bormer, O. P. Immunoassays for carcinoembryonic antigen: specificity and interferences. Scand. J. Clin. Lab Invest 53, 1-9 (1993).
61. Nap, M. et al. Specificity and affinity of monoclonal antibodies against carcinoembryonic antigen. Cancer Res. 52, 2329-2339 (1992).
62. Soletormos, G., Nielsen, D., Schioler, V., Skovsgaard, T., & Dombernowsky, P. Tumor markers cancer antigen 15.3, carcinoembryonic antigen, and tissue polypeptide antigen for monitoring metastatic breast cancer during first-line chemotherapy and follow-up. Clin. Chem. 42, 564-575 (1996).
63. Hostetter, R. B. et al. Carcinoembryonic antigen as a selective enhancer of colorectal cancer metastasis. J. Natl. Cancer Inst. 82, 380-385 (1990).
64. Behrens, J. The role of cell adhesion molecules in cancer invasion and metastasis. Breast Cancer Res. Treat. 24, 175-184 (1993).
65. Vihinen, P., Ala-aho, R., & Kahari, V. M. Matrix metalloproteinases as therapeutic targets in cancer. Curr. Cancer Drug Targets. 5, 203-220 (2005).
66. Leppa, S., Saarto, T., Vehmanen, L., Blomqvist, C., & Elomaa, I. A high serum matrix metalloproteinase-2 level is associated with an adverse prognosis in node-positive breast carcinoma. Clin. Cancer Res. 10, 1057-1063 (2004).

1. A method of screening for, diagnosing or detecting breast cancer in a subject comprising the steps:
(a) determining a level of a biomarker product in a sample from the subject wherein the biomarker is selected from the group consisting of ALCAM, BCAM and a combination thereof, wherein the ALCAM is cleaved, secreted, released or shed from cells; and
(b) comparing the level of each biomarker product in the sample with a control; wherein detecting an increased level of the biomarker product in the sample compared to the control is indicative of breast cancer in the subject.

2. The method according to claim 1 for predicting prognosis of a subject having or suspected of having breast cancer comprising:
(a) determining a level of a biomarker product in a sample from the subject, wherein the biomarker is selected from ALCAM, BCAM and a combination thereof, wherein the ALCAM is cleaved, secreted, released or shed from cells, and
(b) comparing the level of each biomarker with a reference level associated with a disease outcome, the disease outcome being good prognosis, or poor prognosis, wherein the disease outcome associated with the reference level most similar to the level of each biomarker in the sample is the predicted prognosis.

3. The method according to claim 1 for monitoring the therapeutic response of a subject with breast cancer comprising the steps:
(a) determining a level of biomarker product in a first sample of the subject, the biomarker selected from the group consisting of ALCAM, BCAM and a combination thereof, wherein the ALCAM is cleaved, secreted, released or shed from cells;
(b) determining the level of biomarker product in a subsequent sample of the subject, the subsequent sample taken subsequent to the subject receiving a breast cancer treatment or therapy; and
(c) comparing the level of the biomarker product in the first sample to the level of the biomarker product in the subsequent sample,
wherein an increase in the level of the biomarker product is indicative of treatment failure or a negative therapeutic response.

4. The method of claim 2 wherein an increase in ALCAM and/or BCAM compared to the reference level is indicative of poor prognosis.

5-12. (canceled)

13. The method according to claim 1, wherein the level of biomarker product determined comprises extracellular biomarker product.

14. The method of claim 1 wherein the increase in the level of ALCAM and/or BCAM in the sample is at least 20% or at least 25% compared to the control.

15. (canceled)

16. The method of claim 1, wherein the increased level of ALCAM biomarker product indicative of breast cancer is greater than about an 80%, 90% and/or 90-95% specificity cut-off or wherein the increased level of BCAM biomarker product indicative of breast cancer is greater than about an 80%, 90% and/or 90-95% specificity cut-off.

17. (canceled)

18. The method of claim 1, further comprising in step (a) determining a level of at least one additional biomarker product associated with breast cancer wherein the at least one additional biomarker product associated with breast cancer comprises a MUC-1 and/or a CEA biomarker product.

19. (canceled)

20. The method of claim 18 wherein the level of MUC-1 biomarker product is determined by determining the level of CA 15-3 and/or BR 27.29.

21-22. (canceled)

23. The method of claim 20, wherein the CA15-3 level in the sample is normal and/or less than about 30 U/ml.

24-28. (canceled)

29. The method according to claim 1, wherein the breast cancer detected is an early stage breast cancer.

30. The method of claim 1, wherein the breast cancer is non-invasive, metastatic, invasive ductal carcinoma, invasive lobular carcinoma, luminal subtype, basal A-like subtype, ER+, PgR+, ER−, PgR−, PTEN−, Her2/neu amplified, and/or erbB2 amplified.

31. The method according to claim 3 wherein the therapy is chemotherapy or a test therapy.

32. (canceled)

33. The method according to claim 1, wherein the biomarker product is a polypeptide and wherein the polypeptide product comprises cleaved, secreted, released or shed polypeptide.

34-41. (canceled)

42. An immunoassay device for use in the method according to claim 1 for detecting a biomarker comprising a capture antibody immobilized on a solid support, wherein the capture antibody binds a biomarker, the biomarker selected from the group consisting of ALCAM, BCAM and a combination thereof; and a detecting antibody.

43-44. (canceled)

45. The immunoassay device of claim 42, wherein the immunoassay device has a biomarker detection limit of at least 0.05 μg/L and optionally a biomarker detection dynamic range of up to 10 μg/L.

46-50. (canceled)

51. A composition for use in determining the level of biomarker product according to claim 1 comprising an agent, that binds an ALCAM biomarker and/or an agent that binds a BCAM biomarker, wherein the ALCAM is cleaved, secreted, released or shed from cells.

52. (canceled)

53. The composition of claim 51 further comprising an agent that binds a MUC-1 and/or CEA biomarker product optionally wherein the agent that binds the MUC-1 biomarker product comprises an agent that binds CA 15-3, and/or an agent that binds BR 27.29.

54. (canceled)

55. A kit for use in the method according to claim 1 for detecting a biomarker comprising:
(a) an agent that binds a biomarker product selected from the group consisting of ALCAM or BCAM and a combination thereof wherein the ALCAM is cleaved, secreted, released or shed from cells; and
(b) instructions for use.

56-57. (canceled)

58. The kit of claim 55 further comprising an agent that binds a MUC-1 and/or CEA biomarker product, optionally wherein the agent that binds MUC-1 binds CA 15-3 or BR 27.29.

59-62. (canceled)
